SEQUENCE LISTING (12 sequences)

SEQ ID NO: 1 - DNA, 618 bp, Hormaphis cornu
atgaatgtttcatctaaaatttgtctaaaattatttttaataatacaaattgtaataaaatgtgttaaatgcccgtcatcatttgaaattgaagtgaatttgtgtgttgatgatattcgcaccgagttttgtattaccgataataataggactaagtttacgaataaactaccggagtgggcatttaaaaatagaagttcttccatgttgaccacatatgcctcgaaaaaatatttactccatttaatgactcagttagtcgtggacgaaaacatgccaaacctattacaatattgtgatgataaagttaaggatctttcaggaatcgataaaaccttgcatcataagaagtgcaaagatgaattgactaacgtctttaaagaagacaacagcgtaaaggaaattgcagagtttgcgtttactgaactaagtgttgctgaaattaatttactgtctggcacaaatattgcaagagcatattattttaaaatgtttgactatctttcatttaaaagtgagcatcagacacctcataaattttgtcgagccctagtacctaaaataatagaacttaaacaattgaatttgaatgaagaacctgcatctagtagcggccat

SEQ ID NO: 2 - protein, 206 aa, Hormaphis cornu
MNVSSKICLKLFLIIQIVIKCVKCPSSFEIEVNLCVDDIRTEFCITDNNRTKFTNKLPEWAFKNRSSSMLTTYASKKYLLHLMTQLVVDENMPNLLQYCDDKVKDLSGIDKTLHHKKCKDELTNVFKEDNSVKEIAEFAFTELSVAEINLLSGTNIARAYYFKMFDYLSFKSEHQTPHKFCRALVPKIIELKQLNLNEEPASSSGH

SEQ ID NO: 3 - DNA, 546 bp, Artificial Sequence
  Modified
ccgtcatcatttgaaattgaagtgaatttgtgtgttgatgatattcgcaccgagttttgtattaccgataataataggactaagtttacgaataaactaccggagtgggcatttaaaaatagaagttcttccatgttgaccacatatgcctcgaaaaaatatttactccatttaatgactcagttagtcgtggacgaaaacatgccaaacctattacaatattgtgatgataaagttaaggatctttcaggaatcgataaaaccttgcatcataagaagtgcaaagatgaattgactaacgtctttaaagaagacaacagcgtaaaggaaattgcagagtttgcgtttactgaactaagtgttgctgaaattaatttactgtctggcacaaatattgcaagagcatattattttaaaatgtttgactatctttcatttaaaagtgagcatcagacacctcataaattttgtcgagccctagtacctaaaataatagaacttaaacaattgaatttgaatgaagaacctgcatctagtagcggccat

SEQ ID NO: 4 - DNA, 549 bp, Artificial Sequence
  Modified
atgccgtcatcatttgaaattgaagtgaatttgtgtgttgatgatattcgcaccgagttttgtattaccgataataataggactaagtttacgaataaactaccggagtgggcatttaaaaatagaagttcttccatgttgaccacatatgcctcgaaaaaatatttactccatttaatgactcagttagtcgtggacgaaaacatgccaaacctattacaatattgtgatgataaagttaaggatctttcaggaatcgataaaaccttgcatcataagaagtgcaaagatgaattgactaacgtctttaaagaagacaacagcgtaaaggaaattgcagagtttgcgtttactgaactaagtgttgctgaaattaatttactgtctggcacaaatattgcaagagcatattattttaaaatgtttgactatctttcatttaaaagtgagcatcagacacctcataaattttgtcgagccctagtacctaaaataatagaacttaaacaattgaatttgaatgaagaacctgcatctagtagcggccat

SEQ ID NO: 5 - protein, 182 aa, Artificial Sequence
  Modified
PSSFEIEVNLCVDDIRTEFCITDNNRTKFTNKLPEWAFKNRSSSMLTTYASKKYLLHLMTQLVVDENMPNLLQYCDDKVKDLSGIDKTLHHKKCKDELTNVFKEDNSVKEIAEFAFTELSVAEINLLSGTNIARAYYFKMFDYLSFKSEHQTPHKFCRALVPKIIELKQLNLNEEPASSSGH

SEQ ID NO: 6 - protein, 183 aa, Artificial Sequence
  Modified
MPSSFEIEVNLCVDDIRTEFCITDNNRTKFTNKLPEWAFKNRSSSMLTTYASKKYLLHLMTQLVVDENMPNLLQYCDDKVKDLSGIDKTLHHKKCKDELTNVFKEDNSVKEIAEFAFTELSVAEINLLSGTNIARAYYFKMFDYLSFKSEHQTPHKFCRALVPKIIELKQLNLNEEPASSSGH

SEQ ID NO: 7 - DNA, 549 bp, Artificial Sequence
  Modified
atgccctcaagttttgagatagaggtcaacctctgtgtagacgacattcgtacagaattttgtatcacggataacaatcgtaccaagtttacgaataaattacctgaatgggcatttaaaaatagaagttcttcaatgctcacaacgtatgcttctaagaaatacctacttcacctaatgacgcaactcgtggtagatgaaaatatgccgaaccttctccagtactgtgatgacaaggtcaaggatttatcaggaatagataagactctccatcataaaaagtgcaaggacgaacttactaacgttttcaaggaagataacagtgtaaaggagatcgccgagttcgcgttcactgaactgtctgttgctgaaatcaatctcttgtccgggacaaacatcgctagagcgtattattttaagatgttcgattacttatccttcaaatcagaacatcagactcctcacaaattttgccgtgcgttagtgccgaagataatagagttaaaacaactcaacttgaatgaggagccggccagtagcagcgggcac

SEQ ID NO: 8 - DNA, 549 bp, Artificial Sequence
  Modified
atgcctagtagtttcgagatagaggtcaacctgtgcgttgatgatattcggactgagttctgcataacagataataatcgcacaaagttcaccaataaactgcctgaatgggcgttcaaaaataggagttccagcatgttgaccacgtatgcctctaaaaaatacctcctgcacctcatgacccagctcgtcgtggatgagaacatgcccaatctgctgcagtactgtgatgacaaggtgaaagatttgtctggtatagacaagaccctgcatcataagaaatgtaaggacgaattgacgaacgtcttcaaagaggataattccgtcaaggagatcgcagagtttgcgtttactgagctgtccgtggccgaaattaacctcctgtcgggtacaaacattgctagagcctattacttcaaaatgttcgattatctctcatttaagagcgaacatcaaaccccacataagttctgtagggccttggtcccaaaaatcatagaactcaagcaacttaatcttaacgaagaaccagcgtctagttctggccac

SEQ ID NO: 9 - protein, 65 aa, Artificial Sequence
  Oligo
TCGTCGGCAGCGTCAGATGTGTATAAGAGACAGTTTTTTTTTTTTTTTTTTTTTTTTTTTTTTVN

SEQ ID NO: 10 - DNA, 33 bp, Artificial Sequence
  Oligo
tcgtcggcagcgtcagatgtgtataagagacag

SEQ ID NO: 11 - DNA, 34 bp, Artificial Sequence
  Oligo
gtctcgtgggctcggagatgtgtataagagacag

SEQ ID NO: 12 - DNA, 39 bp, Artificial Sequence
  Oligo
gtctcgtgggctcggagatgtgtataagagacargrgrg

(19) United States
(12) Patent Application Publication
(10) Pub. No.: US 2022/0119461 A1
(43) Pub. Date: Apr. 21, 2022

(54) UNIQUE APHID POLYPEPTIDES FOR USE IN MODIFYING CELLS

(71) Applicant: HOWARD HUGHES MEDICAL INSTITUTE, CHEVY CHASE, MD (US)

(72) Inventors: David L. STERN, Leesburg, VA (US); Aishwarya KORGAONKAR, Leesburg, VA (US)

(21) Appl. No.: 17/503,062

(22) Filed: Oct. 15, 2021

Related U.S. Application Data
(60) Provisional application No. 63/092,942, filed on Oct. 16, 2020.

(51) Int. Cl. C07K 14/415; C12N 15/64

(52) U.S. Cl. CPC C07K 14/415; C12N 15/64

(57) ABSTRACT

Unique DGC polynucleotides and polypeptides can be used to modify cells, including plant, animal, fungal, and bacterial cells.

### RELATED APPLICATIONS

This application claims priority from U.S. Provisional Application Ser. No. 63/092,942 filed Oct. 16, 2020, the entire disclosure of which is incorporated herein by this reference.

### TECHNICAL FIELD

The presently-disclosed subject matter generally relates to nucleic acid molecules, polypeptide molecules, compositions, and methods for use in modifying metabolism, growth, and/or size of a cell. In particular, certain embodiments of the presently-disclosed subject matter relate to unique aphid polypeptides molecules, related nucleic acid molecules, and use thereof in compositions and methods for modifying cells.

### INTRODUCTION

Parasites manipulate hosts using many molecular mechanisms. Insect galls represent one of the most extreme forms of such inter-species manipulation. Insect-induced galls are intricately patterned, nutrient rich, protective homes (Mani, 1964), and they do not reflect simply unpatterned cellular over-proliferation, as observed for microbial galls like the crown gall induced by Agrobacterium tumefaciens. Each insect species appears to induce a novel gall, even when related insect species attack the same plant, implying that each insect species provides unique instructions to re-program latent plant developmental networks (Cook and Gullan, 2008; Crespi and Worobey, 1998; Dodson, 1991; Leatherdale, 1955; Martin, 1938; Parr, 1940; Plumb, 1953; Stern, 1995; Stone and Cook, 1998).

The mechanisms used by insects to induce galls have not previously been described. At least some gall-inducing insects produce phytohormones (Dorchin et al., 2009; McCalla et al., 1962; Suzuki et al., 2014; Tanaka et al., 2013; Tooker and Helms, 2014; Yamaguchi et al., 2012), although it is not yet clear that insects inject these hormones or if they contribute to gall growth. However, injection of phytohormones alone probably cannot generate the large diversity of species-specific insect galls. In addition, galling insects induce plant transcriptional changes independent of phytohormone activity (Bailey et al., 2015; Bilder and Irvine, 2017; Nabity et al., 2013; Schultz et al., 2019; Shih et al., 2018; Takeda et al., 2019). Thus, given the complex cellular changes required for gall development, insects probably introduce many molecules to control gall development.

Described herein is a unique aphid gene, determinant of gall color (dgc), that is genetically associated with gall color, providing the first example of an insect gene implicated in gall development.

dgc encodes a “BICYCLE protein,” a new family of diverse secreted proteins produced specifically in salivary glands of galling aphids and that are likely injected into plant cells to modify plant physiology, cell biology, and development. BICYCLE proteins may provide an opportunity to harness their power to manipulate physiology, cell biology, and development of cells in desired ways.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes a polynucleotide molecule, which includes a nucleic acid sequence having at least about 75% identity to SEQ ID NO: 3, SEQ ID NO: 4, SEQ ID NO: 7, or SEQ ID NO: 8.

In some embodiments, the polynucleotide molecule includes a nucleic acid sequence having the sequence of SEQ ID NO: 3, SEQ ID NO: 4, SEQ ID NO: 7, SEQ ID NO: 8, or a functional fragment thereof.

In some embodiments, the polynucleotide molecule is optimized for expression in Arabidopsis thaliana and comprising the sequence of SEQ ID NO: 7. In some embodiments, the polynucleotide molecule is optimized for expression in Oryza sativa and comprising the sequence of SEQ ID NO: 8.

The presently-disclosed subject matter also includes vectors containing one or more of the polynucleotide molecules as disclosed herein.

The presently-disclosed subject matter also includes a composition containing a vector as disclosed herein.

The presently-disclosed subject matter also includes a polypeptide molecule, which includes the amino acid sequence of SEQ ID NO: 5, SEQ ID NO: 6, or a functional fragment thereof.

The presently-disclosed subject matter also includes a composition containing a polypeptide molecule as disclosed herein.

The presently-disclosed subject matter also includes a method of modifying a cell, which involves administering to the cell a vector comprising or a polypeptide encoded by a polynucleotide molecule as disclosed herein. In some embodiments, the cell is selected from the group consisting of a plant cell, an animal cell, a fungal cell, and a bacterial cell.

The presently-disclosed subject matter also includes a method of modulating production of anthocyanins or their precursors in a plant, which involves administering to the plant a vector comprising or a polypeptide encoded by a polynucleotide as disclosed herein. In some embodiments of the method the production of anthocyanins or their precursors is reduced. In some embodiments of the method the anthocyanins are selected from peonidin-3,5-diglucoside and malvidin-3,5-diglucoside. In some embodiments of the method, the precursors are elected from cynidin-3,5-diglucoside, delphinidin-3,5-diglucoside, and petunidin-3,5-diglucoside.

The presently-disclosed subject matter also includes a method of modulating expression of a gene in a cell, comprising: administering to the cell a vector comprising or a polypeptide encoded by the polynucleotide molecule as disclosed herein, wherein the gene is selected from: FAOMT-1, FAOMT-2, GSTF11, GSTF12, UGT75C1, UFGT, ACCA, and combinations thereof. In some embodiments of the method, the cell is selected from the group consisting of a plant cell, an animal cell, a fungal cell, and a bacterial cell. In some embodiments of the method, the gene expression is downregulated.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A-1I. Hormaphis cornu aphids drive patterned over-proliferation of plant cells to produce galls of on leaves Hamamelis virginiana. FIGS. 1A-1C: Photographs of the abaxial surfaces of H. virginiana leaves being attacked by first-instar fundatrices of H. cornu. Nymphs gather on the unopened leaf buds (FIG. 1A) and soon after bud break the fundatrices gather near leaf veins (FIG. 1B) and inject material that begins to transform the leaf into a gall (FIG. 1C). Magnified region in blue rectangle of panel (FIG. 1A, right) shows three fundatrices waiting on unopened bud (FIG. 1A, left). FIGS. 1D-1F: Photographs of the adaxial leaves of H. virginiana, showing galls at early (FIG. 1D), middle (FIG. 1E), and late (FIG. 1F) growth stages. FIGS. 1G-1I: Confocal micrographs of sections through a H. cornu gall (FIGS. 1G and 1H) and un-galled H. virginiana leaf (FIG. 1I) stained with Congo red and calcofluor white. Extensive hypertrophy is observed in the mesophyll (yellow arrows) at a considerable distance from the location of the aphid's stylets (blue arrows) (FIG. 1G). The tips of aphid stylets (pink arrow) can be observed within cells of H. virginiana and plant tissue shows evidence of hyperproliferation and periclinal divisions (grey arrows) close to the tips of stylets and the termini of stylet sheaths (FIG. 1H). Periclinal divisions are observed in both spongy and palisade mesophyll cells during gall development, but never in ungalled leaf tissue (FIG. 1I). Plant tissue is presented in the aphid's frame of reference, with abaxial leaf surface up.

FIGS. 2A-2E. A genome-wide association study (GWAS) identifies variation within a novel aphid gene associated with gall color. FIG. 2A: A GWAS of fundatrices isolated from 43 green and 47 red galls identified a small region on chromosome 1 of the H. cornu genome that is strongly associated with gall color. Red line indicates FDR=0.05. Colors of points on chromosomes are arbitrary. FIGS. 2B-2D: Resequencing 800 kbp of Chromosome 1 centered on the most significant SNPs from the original GWAS to approximately 60× coverage identified 11 spatially clustered SNPs significantly associated with gall color located within the introns and upstream of Horco_g16073, which was named dgc (FIG. 2D). (Some SNPs are closely adjacent and cannot be differentiated at this scale.) Significant SNPs are indicated with orange vertical lines in (FIG. 2D). FIG. 2E: Genotypes of all 11 SNPs associated with gall color from an independent sample of aphids from 435 green and 431 red galls. All 11 SNPs are strongly associated with gall color (P<10⁻¹⁹²), and a cluster of 5 SNPs in a 61 bp region are most strongly associated with gall color as illustrated by LOD scores shown at bottom of plot. Histogram of frequencies of each genotype within gall color is shown on the right.

FIGS. 3A-3H. dgc is the only strongly differentially expressed gene in salivary glands of fundatrices collected from red versus green galls. FIG. 3A: Genome-wide differential expression analysis of H. cornu fundatrix salivary gland transcripts from individuals heterozygous for dgc^{Red}/dgc^{Green} (Red; N=15) versus homozygous for dgc^{Green} (Green; N=7) illustrated as a volcano plot. dgc is strongly downregulated in dgc^{Red}/dgc^{Green} fundatrices. FIG. 3B: Salivary gland expression of genes in the paralog cluster that includes dgc^{Red}/dgc^{Green}. Gene models of the paralogous genes are shown at the top and expression levels normalized by mean expression across all paralogs is shown below. Only dgc is strongly downregulated in this gene cluster between individuals carrying dgc^{Red}/dgc^{Green} (Red) versus dgc^{Green}/dgc^{Green} (Green) genotypes. FIG. 3C: Genome-wide differential expression analysis of H. virginiana transcripts isolated from galls made by fundatrices heterozygous for dgc^{Red}/dgc^{Green} (Red; N=17) versus homozygous for dgc^{Green} (Green; N=22) illustrated as a volcano plot. Only eight genes are differentially expressed at FDR<0.05, and all are overexpressed in red galls. The seven most strongly differentially expressed genes that encode anthocyanin biosynthetic enzymes (FAOMT-1=Hamvi_23591; FAOMT-2=Hamvi_7147; GSTF11=Hamvi_134919; GSTF12=Hamvi_109682; UGT75C1=Hamvi_14194; UFGT=Hamvi_22774; ACCA=Hamvi_97071). FIG. 3D. Expression levels, in counts per million reads, of the seven anthocyanin biosynthetic genes overexpressed in red galls, in green (green) and red (red) galls and ungalled leaves (blue). Each data point is from a separate genome-wide RNA-seq sample. FIG. 3E: Simplified diagram of the anthocyanin biosynthetic pathway. Enzyme classes upregulated in red galls are shown in purple font. The two terminal anthocyanins that generate the red color in galls, peonidin-3,5-diglucoside and malvidin-3,5-diglucoside, are shown in bold font, and three precursor molecules found in red galls are shown in bold italic font. Anthocyanin names are abbreviated (dg=3,5-diglucoside). FIG. 3F: Photos of cross-sections of green (top left) and red (top right) and the pigments extracted from green and red galls (below). FIG. 3G: UHPLC-DAD chromatograms at 520 nm of extract from red (red line) and green (green line) galls. FIG. 3H: Overlaid UHPLC-MS chromatograms of green (top) and red (middle) gall extracts and authentic standards (bottom). Each pigment is indicated with a different color: green=delphinidin-3,5-diglucoside (m/z=627.1551); black=cyanidin-3,5-diglucoside (m/z=611.1602); blue=petunidin-3,5-diglucoside (m/z=641.1709); purple=peonidin-3,5-diglucoside (m/z=625.1768); and red=malvidin-3,5-diglucoside (m/z=655.1870). Anthocyanin names are abbreviated (dg=3,5-diglucoside). Eighty-seven percent of pigment in red galls was found in the two anthocyanins peonidin-3,5-diglucoside and malvidin-3,5-diglucoside.

FIGS. 4A-4F. Bicycle genes are salivary gland enriched transcripts of gall-associated H. cornu generations. FIG. 4A: Diagram of life cycle of H. cornu. H. cornu migrates annually between H. virginiana (blue line) and Betula nigra (pink line) and the gall is produced only in the spring on H. virginiana (brown line). Each nymph of the first generation (G1, the fundatrix) hatches from an over-wintering egg and initiates development of a single gall. Her offspring (G2) feed and grow within the gall and develop with wings, which allows them to fly to B. nigra in late spring. For three subsequent generations (G3-G5) the aphids develop as small, coccidiform morphs on B. nigra. In the fall, aphids develop with wings (G6), fly to H. virginiana plants, and deposit male and female sexuals (G7), the only generation possessing meiotic cells. The sexuals feed and complete development on the senescing leaves of H. virginiana. As adults they mate and the females deposit eggs that overwinter and give rise to fundatrices the following spring. FIG. 4B: Differential expression of fundatrix versus sexual salivary glands with only genes significantly upregulated in fundatrix salivary glands marked with colors, shown as a volcano plot. Genes with and without homologs in public databases are labeled as “Annotated” (yellow) and “Not Annotated” (purple), respectively. FIG. 4C: Hierarchical clustering of unannotated salivary-gland specific genes reveals one large cluster of bicycle genes, one small cluster of CWG genes, and many largely unique, unclustered genes. FIG. 4D: Bicycle (purple) and remaining unannotated (yellow) genes labelled on a differential expression volcano plot illustrates that, on average, bicycle genes are the most strongly differentially expressed genes expressed specifically in fundatrix salivary glands. FIG. 4E: Bicycle genes are expressed at highest levels in the salivary glands of the fundatrix and mostly decline in expression during subsequent generations. (Sample sizes: G1 (N=20); G2 (N=4); G5 (N=6); and G7 (N=6). FIG. 4F: Amino-acid logo for predicted BICYCLE proteins. Sequence alignment used for logo was filtered to highlight conserved positions. Most genes encode proteins with an N-terminal signal sequence and a pair of conserved cysteine-tyrosine-cysteine motifs (CYC).

FIGS. 5A-5I. Supporting genetic evidence that the 11 SNPs located near dgc are the only genetic variants strongly associated with gall color. FIGS. A-D: The first two components of a principal components analysis (PCA) of genome-wide variation (FIG. 5A) and of variation in an 800 kbp window centered on dgc (FIG. 5C) reveal no large scale association of genetic variation with gall color. The top ten principal components of the genome-wide (FIG. 5B) and targeted (FIG. 5D) PCA each explain less than 2% and 5%, respectively, of the genetic variance. FIGS. 5E-5I: Distribution of mapped, unpaired Illumina sequencing reads provides no evidence for common large-scale genomic aberrations in aphids from either green (above) or red (below) galls. Discordant reads from fundatrices making red (FIG. 5E) and green (FIG. 5F) galls from the entire ˜800 kbp resequenced region are rare and are not specifically associated with gall color (FIG. 5G). Close examination of the genomic region harboring SNPs strongly associated with gall color (red vertical lines) shows that discordant reads are rare and not linked to the associated SNPs. Discordant reads do not map preferentially anywhere in the genome (FIG. 5I).

FIGS. 6A-6C. Genotypes of SNPs in the dgc gene region. FIG. 6A: Gene models for the region ˜40.475-40.52 of Chromosome 1. dgc is labelled in red, other bicycle genes are labelled in purple. FIG. 6B: Genotypes for each of the 90 individuals (42 from green galls, 48 from red galls; 4 low coverage individuals were excluded) from the original GWAS inferred from approximately 60× sequencing coverage for this region. Each row represents the genotypes at each of 138 SNPs. Genotypes are color coded as homozygous reference alleles (blue), heterozygous (orange), homozygous non-reference allele (purple). FIG. 6C: Frequencies of each genotype at each variable position.

FIGS. 7A-7B. An alignment-free association test identifies most of the same associated SNPs as the GWAS. FIG. 7A: Distribution of significant kmers associated with gall color detected by the alignment-free association test HAWK (Rahman et al. 2018) in the approximately 800 kb re-sequenced region centered on dgc (approximately 40.48-40.5 Mbp). The most significant kmers are found only located within and close to dgc. Non-significant kmers are not shown. FIG. 7B: Region from approximately 40.475-40.51 of plot (A), showing the finer-scale distribution of significant kmers. The red lines indicate SNPs detected in the original GWAS. All of the GWAS SNPs are confirmed by the alignment-free association test. The absence of significant kmers elsewhere in this region suggests that there are no genome rearrangements of transposon insertions that are associated with gall color.

FIG. 8. Predicted amino-acid sequence of g16073. Several secondary structure predictions are shown, including SignalP 5.0 prediction (Almagro Armenteros et al. 2019b) of an N-terminal secretion signal from positions 1-24 (bold, underlined) and jpred4 secondary structure prediction (Drozdetskiy et al. 2015) below primary sequence (H=helical, E=extended). Cysteines and tyrosines are colored red and blue, respectively, and the two CYC motifs are underlined. Orange triangles above protein sequence indicate intron-exon boundaries.

FIGS. 9A-9B. Linkage disequilibrium in dgc gene region. FIG. 9A: Gene models for the region ˜40.475-40.52 of Chromosome 1. dgc is labelled in red, other bicycle genes are labelled in purple. FIG. 9B: Linkage disequilibrium between all sites. Strong linkage disequilibrium is observed between the 11 SNPs linked to gall color (labelled as “Significant GWAS SNPs” above plot), but not between these SNPs and other variable sites.

FIGS. 10A-10D. No evidence for long-distance linkage disequilibrium among variable sites in an 800 kbp region centered on dgc. FIG. 10A: Gene models for the region ˜40.1-40.9 of Chromosome 1. dgc is labelled in red, other bicycle genes are labelled in purple. FIG. 10B: Genotypes for each of the 94 individuals (N from green galls, M from red galls) from the original GWAS inferred from approximately 60× sequencing coverage. Each row represents the genotypes at each of N SNPs. Genotypes are color coded as homozygous reference alleles (blue), heterozygous (orange), homozygous non-reference allele (purple). FIG. 10C: Frequencies of each genotype at each variable position. FIG. 10D: Linkage disequilibrium between all sites. Strong linkage disequilibrium is observed between the 11 SNPs linked to gall color (labelled as “Significant GWAS SNPs” above plot), but not between these SNPs and other variable sites.

FIGS. 11A-11D. Expression of dgc. FIG. 11A: Expression of dgc in salivary glands, whole bodies, or carcasses (body minus salivary glands) throughout the H. cornu life cycle. Salivary glands were dissected from multiple nymphal stages and adults of four generations representing major morphs of the life cycle, G1 (fundatrix), G2, G5, and sexuals. dgc is expressed at highest levels in salivary glands of fundatrices. dgc expression declines in salivary glands through the instars of G2 animals and later generations and was not detected in salivary glands of sexuals. Expression observed in full bodies of G1 animals (fundatrices) probably reflect expression in the salivary glands and expression was not observed in carcasses. FIG. 11B: MD plot showing that dgc is the only gene differentially expressed in salivary glands of fundatrices sampled from red versus green galls and is strongly downregulated. The mean expression level of dgc illustrates that it is among the most highly expressed genes in salivary glands. Aphids from red and green galls were genotyped and carried dgc^{Red}/dgc^{Green} and dgc^{Green}/dgc^{Green} genotypes at all 11 SNPs associated with gall color, respectively. FIG. 11C: Expression levels of genes in the bicycle gene paralog group that includes dgc from a single individual with a dgc^{Green}/dgc^{Green} genotype at all 11 SNPs that inhabited a red gall. dgc is expressed at similar levels as Horco_16072, similar to the pattern observed for individuals that make green galls (FIG. 4B). Thus, it is likely that this individual, and possibly the remaining rare fundatrices that induce red galls with dgc^{Green}/dgc^{Green} genotypes, do not do so through reduction in dgc expression, but instead carry genetic variation at one or more other genes that induce red galls. FIG. 11D: Number of fundatrix salivary gland RNA-seq reads containing alternative dgc exonic SNPs at two genomic location for aphids collected from green (blue font) and red (red font) galls. Four out of eight green and one out of two red samples deviate from the expectation of equal expression from both alleles in an individual. Thus, there is no evidence for allelic imbalance between fundatrices making green and red galls. In addition, both alternative alleles in red samples are expressed at substantially lower levels than the same alleles in green samples. Thus, a single copy of the SNPs that repress dgc expression (FIG. 4B) appear to repress dgc from both alleles in heterozygous individuals.

FIGS. 12A-12E. MS/MS spectra of anthocyanins detected in the red gall extract, which clearly demonstrate the aglycone product ions previously reported for cyanidin-3,5-diglucoside (FIG. 12A), delphinidin-3,5-diglucoside (FIG. 12B), malvidin-3,5-diglucoside (FIG. 12C), peonidin-3,5-diglucoside (FIG. 12D), and petunidin-3,5-diglucoside (FIG. 12E).

FIGS. 13A-13B. Genome-wide differential expression analysis of H. virginiana galls versus leaves. FIG. 13A: Genome-wide differential expression analysis of H. virginiana transcripts isolated from galls (N=36) versus leaves (N=17). Approximately 60% of expressed genes are differentially expressed between gall and leaf tissue at FDR<0.05. FIG. 13B: Gene ontology analysis of GO terms down (left) and up-regulated (right) in galls, presented as volcano plots. Genes involved in cell division and morphogenesis were strongly upregulated in galls and genes involved in photosynthesis were strongly down-regulated in galls.

FIGS. 14A-14H. Further analysis of H. cornu fundatrix salivary gland enriched and bicycle genes. FIGS. 14A-14B: Differential expression comparing gene expression in fundatrix salivary glands versus fundatrix whole bodies (FIG. 14A) and salivary glands of fundatrices versus sexuals (FIG. 14B) shown as volcano plots. Both comparisons reveal that a large number of genes are strongly over-expressed in fundatrix salivary glands. Intersection of the genes over-expressed in these two comparisons yielded the focal collection of genes over-expressed specifically in fundatrix salivary glands (FIG. 6B). Genes differentially over- or under-expressed at FDR<0.05 are shown in blue or red, respectively. FIG. 14C: Volcano plot of Gene Ontology over-representation analysis of “Annotated” genes from FIG. 6B showing KEGG pathway terms for biological processes that were over-represented with a raw P value<0.05. No GO terms were significantly under-represented with a raw P value<0.05. Analyses performed with WebGestalt (Liao et al. 2019). FIG. 14D: MD plot of differential expression results shown in panel (FIG. 14B) with “Unannotated” and “Annotated” genes with signal peptides over-expressed in fundatrix salivary glands labeled in purple and yellow, respectively. FIG. 14E: Distribution of singleton bicycle genes and paralog clusters in the H. cornu genome. Number of genes per cluster and genomic range is indicating by height and width, respectively, of blue bars above chromosomes. Histogram of number of bicycle genes per paralog cluster is shown in inset. FIG. 14F: Example of part of a paralog cluster of bicycle genes from chromosome 7 of the H. cornu genome, illustrating abundance of small exons in each gene. FIG. 14G: Number of exons per gene versus median exon size for H. cornu bicycle (blue) and non-bicycle (red) genes. Bicycle genes possess an unusually large number of unusually small exons. FIG. 14H: Maximum likelihood phylogenetic tree of H. cornu bicycle gene amino acid sequences reveals extensive sequence divergence of bicycle genes.

FIGS. 15A-15F. Further analysis of unannotated fundatrix salivary gland enriched genes. FIG. 15A: Logo plot of the aligned predicted protein sequences from CWG genes. Sequence alignment used for logo was filtered to highlight conserved positions. FIGS. 15B and 15E: Volcano plots of differential expression of fundatrix versus sexual salivary glands with CWG genes (FIG. 15B) and Unclustered genes (FIG. 15E) labelled in purple for clusters identified through hierarchical clustering (FIG. 6C). (FIGS. 15C and 15F) Expression levels of CWG (FIG. 15C) and Unclustered (FIG. 15F) genes in the salivary glands of individuals across four generations of the H. cornu life cycle. Lines connect the same gene across generations and are color coded by relative expression levels in G1, with blue to red representing most to least strongly expressed, respectively. FIG. 15D: bicycle genes are, on average, the most strongly differentially expressed category of unannotated genes.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes nucleic acid molecules and polypeptide molecules related to a determinant of gall color (DGC) gene and protein, and methods for modifying a plant.

As disclosed herein, a DGC gene is an aphid gene that can produce a DGC Protein in the salivary glands of galling aphids. As described further herein, a number of single nucleotide polymorphisms (SNPs) have been identified that are regulatory, and result in turning off expression of the DGC gene. When the DGC gene is turned off, such that the DGC protein is absent from the saliva of a galling aphid, the result is a plant gall that is red. When the DGC gene is expressed, such that the DGC protein is present in the saliva of the galling aphid, a green gall results in the plant.

In this regard, and with reference to the studies described hereinbelow, DGC affects the anthocyanin production pathway, having an impact on enzyme classes (ACCA, UFGT, FAOMT, GSTF), anthocyanins that generate the red color in galls (peonidin-3,5-diglucoside(dg) and malvidin-dg), and anthocyanin precursor molecules found in red galls (cynidin-dg, delphinidin-dg, petunidin-dg).

The Hormaphis cornu aphid wild type DGC gene has the nucleotide sequence of SEQ ID NO: 1. The Hormaphis cornu aphid wild type DGC protein has the amino acid sequence of SEQ ID NO: 2. Disclosed herein are modified sequences for use in expressing DGC in plants.

The wild type aphid DGC sequences includes a signal secretion sequence, which can be removed to express DGC in the plant while preventing secretion from the cell of the expressed DGC protein. The signal secretion sequence includes about the first 20 amino acids of a bicycle gene protein, such as the DGC gene protein.

In some embodiments, an N-terminal methionine can be added to the modified sequence to provide for proper translation.

In some embodiments, the codons of the nucleotide sequence can be further modified to optimize expression in a particular plant of interest, which can be accomplished by one of ordinary skill in the art using known methods, after studying the present disclosure. In this regard, in some embodiments, one could generate a DNA sequence that contains, in the following order, an enhancer sequence to drive gene expression in a relevant plant tissue, a plant promoter that supports high levels of gene expression, the nucleotide sequence encoding the protein of interest, and finally a plant 3′ UTR that supports high levels of protein translation.

The optimized gene can be expressed in a plant of interest using a vector known by those of ordinary skill in the art, selected with consideration to the plant species and plant organ of interest.

In some embodiments, the codons of the nucleotide sequence and the enhancer, promoter and 3′ UTR sequences could be specified to allow expression in animal, fungal, or bacterial cells.

The presently-disclosed subject matter includes a polynucleotide molecule, comprising a nucleic acid sequence encoding the sequence of SEQ ID NO: 5 or a functional fragment thereof. In some embodiments, the nucleic acid sequence is modified relative to the sequence of SEQ ID NO: 3 for optimized expression in a particular plant species of interest. In some embodiments, the polynucleotide molecule is optimized for expression in Arabidopsis thaliana and comprising the sequence of SEQ ID NO: 7. In some embodiments, the polynucleotide molecule is optimized for expression in Oryza sativa and comprising the sequence of SEQ ID NO: 8.

The presently-disclosed subject matter further includes a polynucleotide molecule, comprising a nucleic acid sequence having at least about 98, 95, 90, 85, 80, 75, 70, 65, 60, 55, 50, 45, 40, 35, 30, or 25% identity to SEQ ID NO: 3, SEQ ID NO: 4, SEQ ID NO: 7, or SEQ ID NO: 8. In some embodiments, the nucleic acid sequence has at least 75% identity to SEQ ID NO: 3, SEQ ID NO: 4, SEQ ID NO: 7, or SEQ ID NO: 8. In some embodiments, the nucleic acid sequence has the sequence of SEQ ID NO: 3, SEQ ID NO: 4, SEQ ID NO: 7, or SEQ ID NO: 8. In some embodiments, the nucleic acid sequence is modified relative to SEQ ID NO: 3 to optimize expression in a particular plant species of interest. Such optimization can be achieved, upon study of this document, by one of ordinary skill.

The presently-disclosed subject matter further includes a vector, comprising a polynucleotide molecule as disclosed herein.

The presently-disclosed subject matter further includes a composition comprising a polypeptide molecule comprising the sequence of SEQ ID NO: 5 or SEQ ID NO: 6, formulated for administration to a plant. The presently-disclosed subject matter further includes a composition comprising a polynucleotide molecule as disclosed herein. The presently-disclosed subject matter further includes a composition comprising a vector as disclosed herein.

The presently-disclosed subject matter includes methods of modifying a plant. In some embodiments, the method involves administering to the plant a polynucleotide molecule, a vector, and/or a composition as disclosed herein.

The presently-disclosed subject matter further includes a method of modulating production of anthocyanins or their precursors in a plant, comprising: administering to the plant a polynucleotide molecule, a vector, and/or a composition as disclosed herein. In some embodiments of the method, the production of anthocyanins or their precursors is reduced. In some embodiments of the method, the anthocyanins are selected from peonidin-3,5-diglucoside and malvidin-3,5-diglucoside. In some embodiments of the method, the precursors are elected from cynidin-3,5-diglucoside, delphinidin-3,5-diglucoside, and petunidin-3,5-diglucoside.

The presently-disclosed subject matter further includes a method of modulating expression of a gene in a plant, comprising: administering to the plant a polynucleotide molecule, a vector, and/or a composition as disclosed herein, wherein the gene is selected from: FAOMT-1, FAOMT-2, GSTF11, GSTF12, UGT75C1, UFGT, ACCA, and combinations thereof.

In some embodiments of the method, the gene expression is downregulated

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The terms “polypeptide fragment” or “fragment”, when used in reference to a reference polypeptide, refers to a polypeptide in which amino acid residues are deleted as compared to the reference polypeptide itself. A fragment can also be a “functional fragment,” in which case the fragment retains some or all of the activity of the reference polypeptide as described herein.

As used herein, an “activity” of a polypeptide refers to any activity exhibited by the reference full-length BICYCLE protein. Activity of a modified polypeptide can be any level of percentage of activity of the full-length polypeptide.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### EXAMPLES

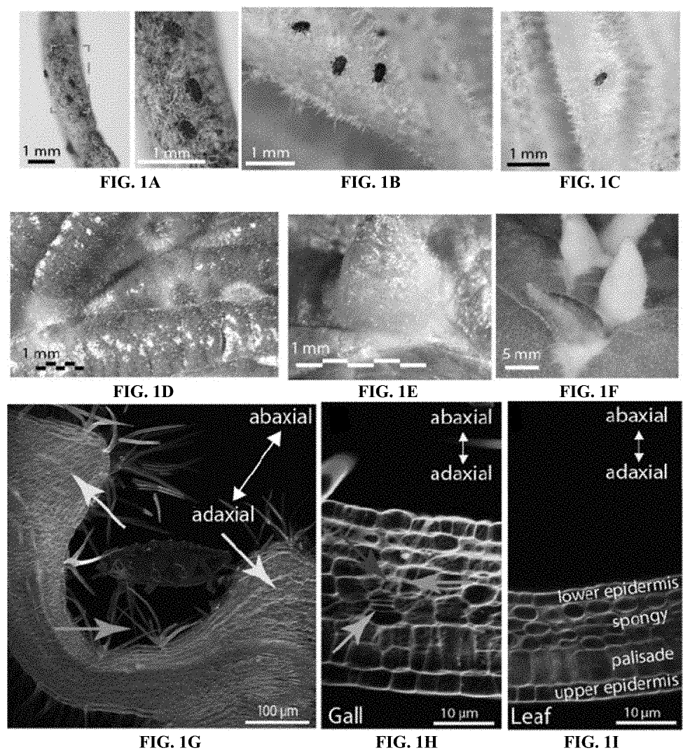

The aphid, Hormaphis cornu, induces galls on the leaves of witch hazel, Hamamelis virginiana, in the Eastern United States (FIGS. 1A-1F). In early spring, each H. cornu gall foundress (fundatrix) probes an expanding leaf with her very thin mouthparts (stylets) (FIGS. 1A-1B). Her stylets pierce mesophyll cells (FIGS. 1G-1H), where aphids do not normally feed, and inject fluid directly into plant cells and perhaps into inter-cellular spaces (Lewis and Walton, 1947, 1958). Plant cells near the injection sites over-proliferate through periclinal cell divisions (FIG. 1H), a pattern of cytokinesis not otherwise observed in leaves at this stage of development (FIG. 1I), and cells elongate, causing thickening and apical expansion of leaf tissue (FIGS. 1B-1E).

After several days, the basal side of the gall encloses the fundatrix and the gall continues to grow apically and laterally, providing the fundatrix and her offspring with protection and abundant food. After several weeks, the basal side of the gall opens to allow aphids to remove excreta (honeydew) and molted nymphal skins from the gall and, eventually, to allow winged migrants to depart. Continued gall growth requires the constant presence of the fundatrix and gall tissue dies in her absence (Lewis and Walton, 1958; Rehill and Schultz, 2001), suggesting that the fundatrix must continuously inject material into plant tissue to overcome plant defenses.

A Natural Gall Color Polymorphism is Linked to Regulatory Variation in a Novel Aphid Gene, Determinant of Gall Color

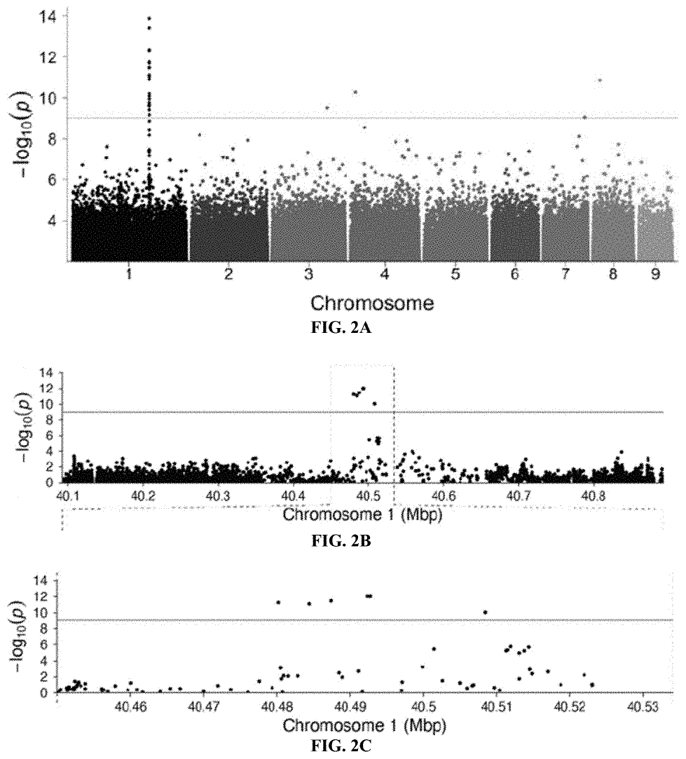

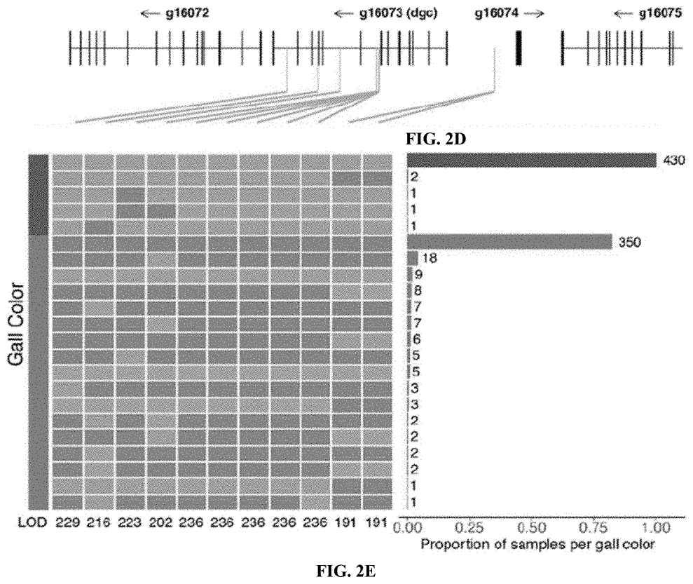

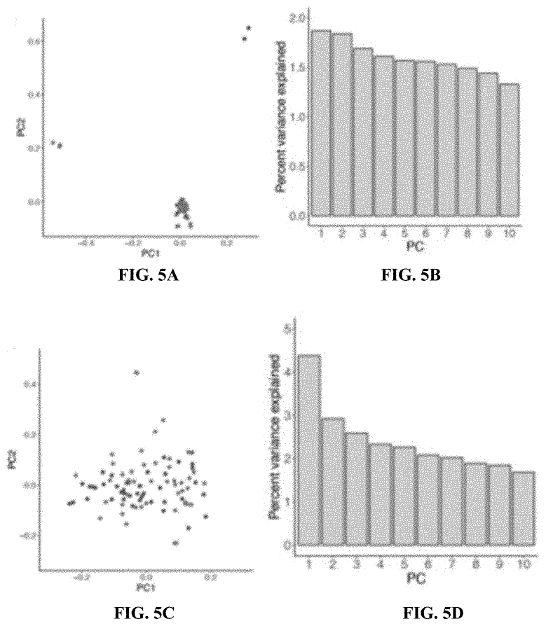

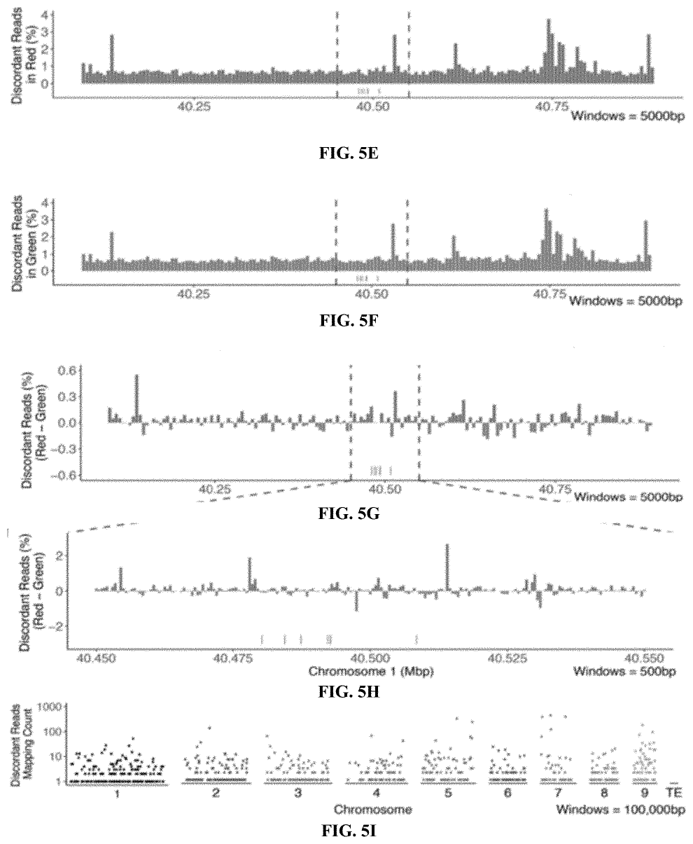

Populations of H. cornu include approximately 4% red galls and 96% green galls (FIG. 1F). It was inferred that the gall color polymorphism results from differences among aphids, rather than underlying differences in the leaves on which they occur, because red and green galls can be found adjacent to each other on the same leaf (FIG. 1F). A genome-wide association study (GWAS) was peformedon fundatrices isolated from 43 green galls and 47 red galls by resequencing their genomes to approximately 3× coverage. There is no evidence for genome-wide differentiation of samples from red and green galls, suggesting that individuals making red and green galls were sampled from a single interbreeding population (FIGS. 5A-5D). Many strongly-associated SNPs were observed near 40.5 Mbp on Chromosome 1 (FIG. 2A). Approximately 800 kbp flanking these SNPs was re-sequenced to approximately 60× coverage and identified 11 single-nucleotide polymorphisms (SNPs) within the introns and upstream of gene Horco_16073 strongly associated with gall color (FIGS. 2B-2D). There is no evidence that large scale chromosomal aberrations are associated with gall color.

Since GWAS can sometimes produce spurious associations, an independent replication study was performed and it was found that all 11 SNPs were highly significantly associated with gall color in fundatrices isolated from 435 green and 431 red galls (LOD=191-236; FIG. 2E). All fundatrices from green galls were homozygous for the ancestral allele at 9 or more of these associated SNPs (FIG. 2E). In contrast, 98% of fundatrices from red galls were heterozygous or homozygous for derived alleles at 9 or more SNPs (FIG. 2E). This pattern suggests that alleles contributing to red gall color are genetically dominant to alleles that generate green galls. No SNPs displayed perfect association with gall color (FIG. 2E), suggesting that multiple derived SNPs at Horco_16073 can confer red color to galls.

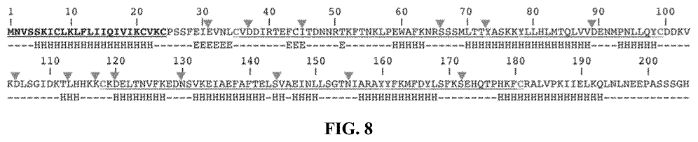

Based on these genetic associations and further evidence presented below, the name determinant of gall color (dgc) to Horco_16073 was assigned. dgc encodes a predicted protein of 23 kDa with an N-terminal secretion signal sequence (FIG. 8). The putatively secreted portion of the protein shares no sequence homology with any other reported proteins.

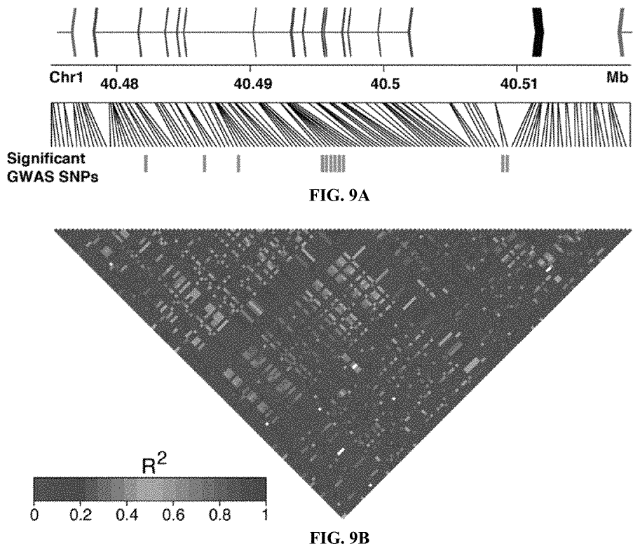

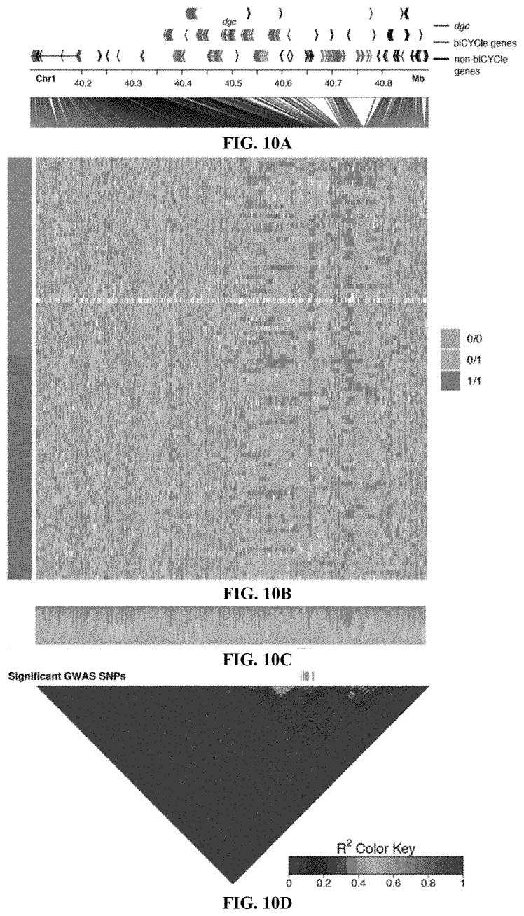

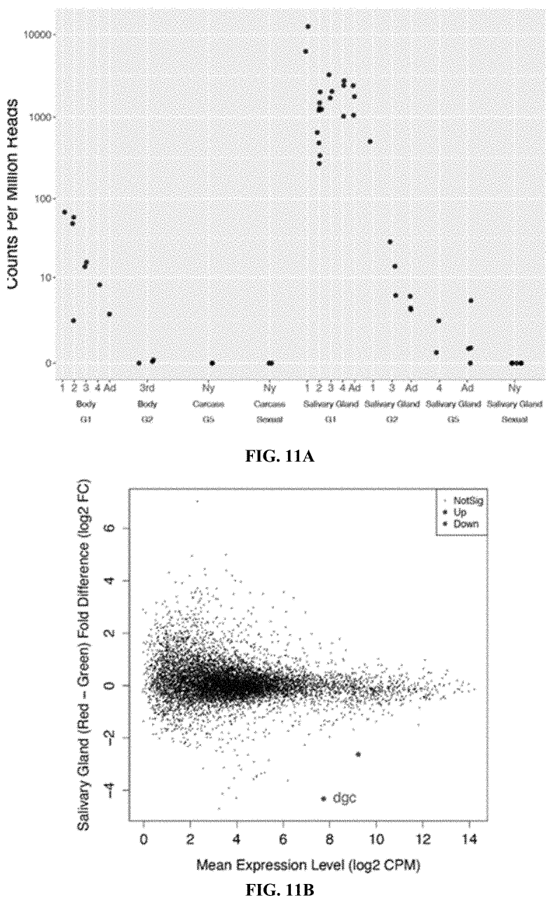

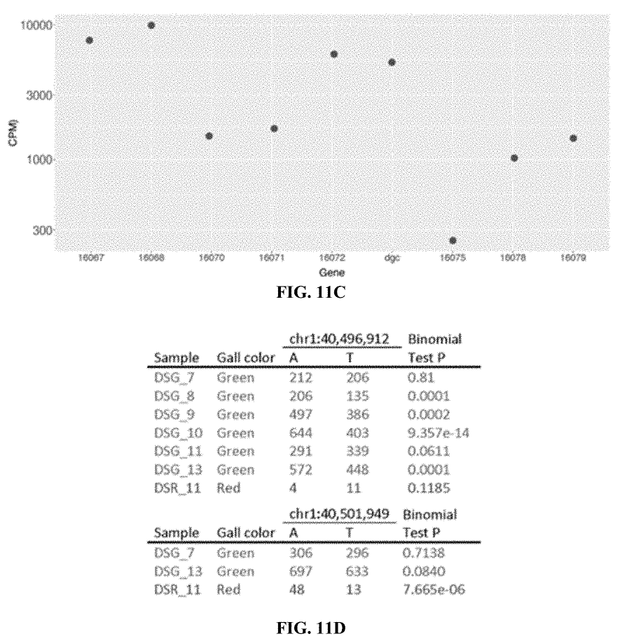

Most SNPs associated with green or red galls were found in one of two predominant haplotypes (FIG. 2E) and exhibited strong linkage disequilibrium (LD) (FIG. 9). LD can result from suppressed recombination. However, these 11 SNPs are in linkage equilibrium with many other intervening and flanking SNPs (FIGS. 9-10). Also, multiple observed genotypes are consistent with recombination between these 11 SNPs (FIG. 2E) and no evidence was found for chromosomal aberrations that could suppress recombination (FIGS. 5E-5I). Thus, LD of the 11 dgc SNPs associated with gall color cannot be explained by suppressed recombination. The most parsimonious explanation for this pattern is that the combination of multiple dgc^{Red} SNPs has been maintained by natural selection. Thus, most or all of the 11 derived dgc SNPs contribute to production of red galls and natural selection appears to have favored the combined action of all 11 SNPs.

Chromosomal Aberrations Cannot Explain Association of 11 SNPs with Gall Color

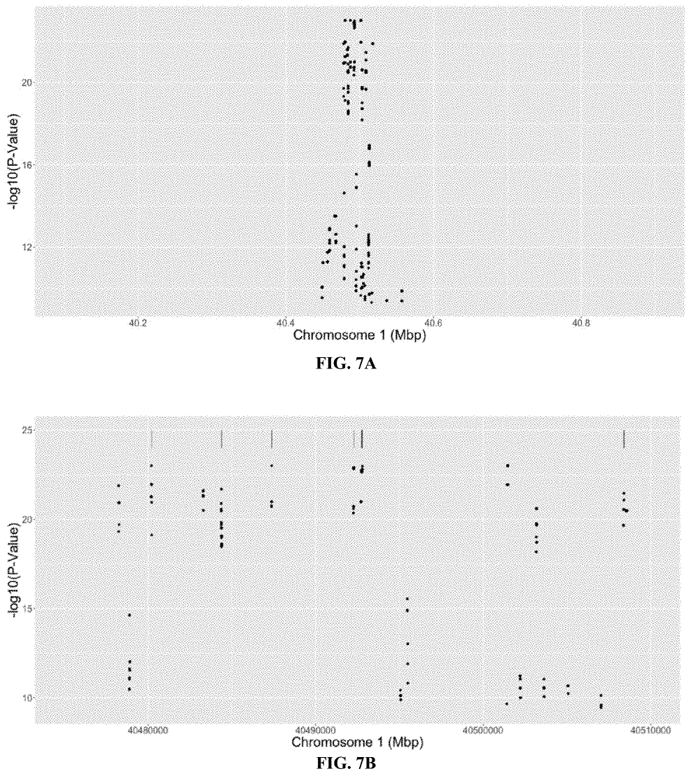

The possibility was considered that DNA aberrations linked to the 11 associated SNPs might explain the red-green gall polymorphism. No evidence was found for large insertions, deletions, or other chromosomal aberrations in this region (FIGS. 5E-5I). In addition, a genome-free association study (Rahman et al. 2018) was performed and strong linkage was found for only for a subset of the 11 SNPs identified in the original GWAS (FIG. 7), further supporting the inference that these SNPs, and not other genetic changes, are associated with gall color.

Gene Ontology Analysis of Plant Genes Differentially Expression in Galls is Consistent with the Observed Cell Biology of Gall Development

Genes associated with cell division were strongly upregulated in galls (FIG. 5H), consistent with the extensive growth observed in gall tissue (FIGS. 1G-I). Genes associated with chloroplasts and photosynthesis were strongly downregulated in gall tissue (FIG. 5I), probably reflecting the reprogramming of plant leaf tissue from nutrient exporters to nutrient sinks (Inbar, Eshel, and Wool 1995). Downregulation of genes related to immune response and multiple defense mechanisms in gall tissue were also observed (FIG. 5I), which may reflect suppression of the plant's defense responses to infection. The pattern of differential expression does not appear to reflect a plant wound response, because wound response genes are not significantly over-expressed in gall tissue (Chi-square P-value=0.367) and are biased toward under-represented.

Regulatory Variants at Dgc Dominantly Silence Dgc Expression

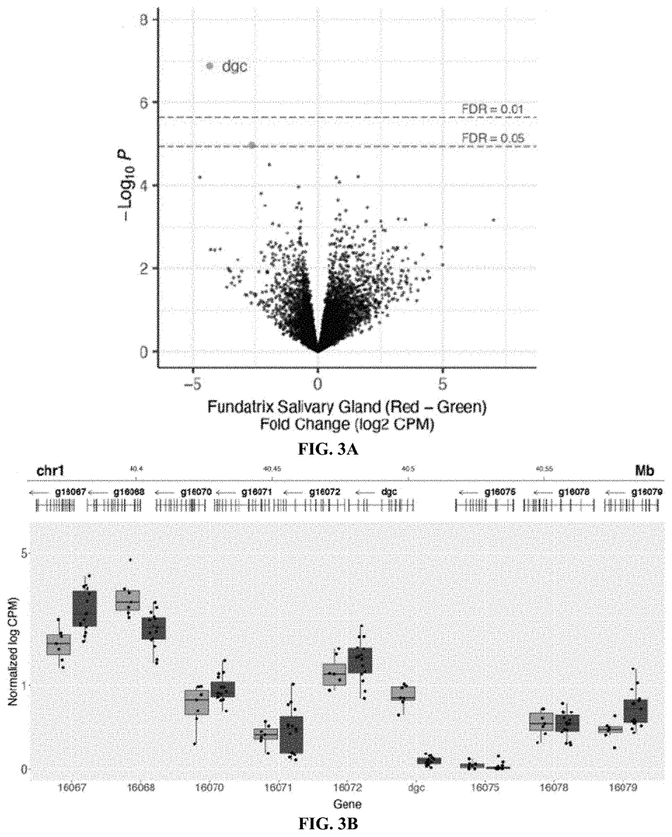

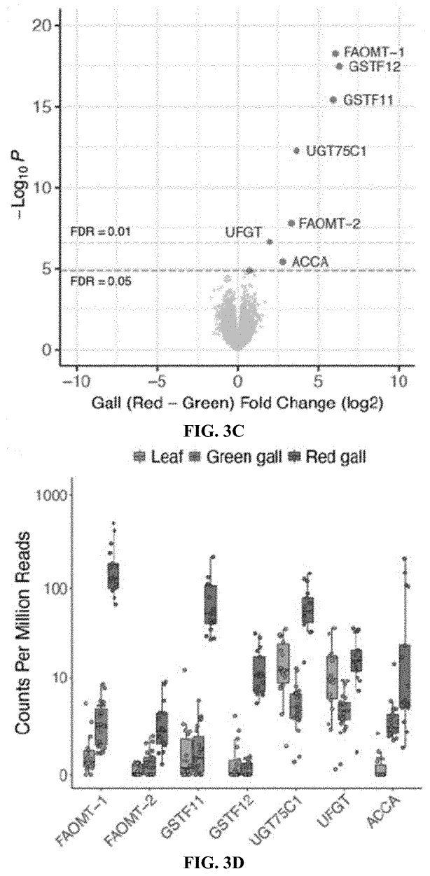

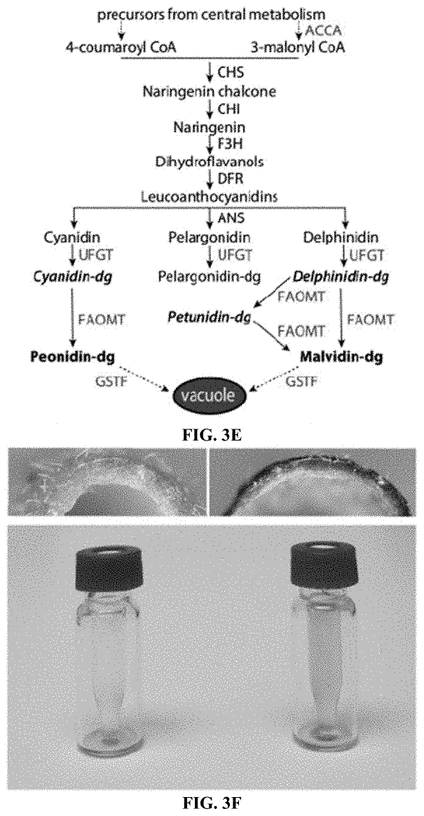

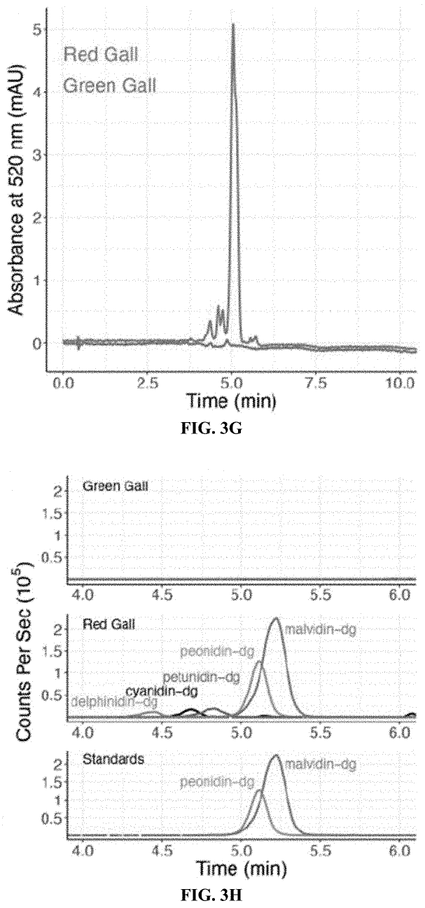

Since all 11 dgc polymorphisms associated with gall color occur outside of dgc exons (FIG. 2D), studies were conducted to determine whether these polymorphisms influence expression of dgc or of any other genes in the genome. It was first determined that dgc is expressed highly and specifically in fundatrix salivary glands and lowly or not at all in other tissues or other life cycle stages (FIG. 11A). RNAseq was then performed on salivary glands from fundatrices with dgc^{Green}/dgc^{Green} or dgc^{Red}/dgc^{Green} genotypes. dgc is the only strongly differentially expressed gene between these genotypes (FIGS. 3A and 11B). Since dgc^{Red} alleles appear to be dominant to the dgc^{Greed} alleles for gall color gall color, dgc transcript was expected to be upregulated in animals with dgc^{Red} alleles. In contrast, it was unexpectedly determined that dgc transcript is almost absent in fundatrices carrying dgc^{Red} alleles (FIG. 3B). That is, red galls are associated with reduced dgc expression in fundatrix salivary glands.

dgc expression is reduced approximately 20-fold in fundatrix salivary glands with dgc^{Red}/dgc^{Green} (27±22.6 CPM, mean±SD) versus dgc^{Green}/dgc^{Green} genotypes (536±352.3 CPM, mean±SD). Thus, dgc^{Red} alleles probably downregulate both the dgc^{Red} and dgc^{Green} alleles in heterozygotes. By examining exonic SNPs in dgc, it was confirmed that the dgc transcript is strongly downregulated from both dgc^{Red} and dgc^{Green} alleles in heterozygotes (FIG. 10D). No systematic transcriptional changes were observed in neighboring genes (FIG. 3B), most of which are dgc paralogs.

High Levels of Dgc Transcription are Associated with Downregulation Specifically of Plant Anthocyanin Genes and Two Anthocyanins

Since red galls are associated with downregulation of only dgc, consideration was given to how the plant responds to changes in this single putative signal. To examine this question, whole-genome differential expression was performed on plant mRNA isolated from galls induced by aphids with dgc^{Red}/dgc^{Green} versus dgc^{Green}/dgc^{Green} genotypes (Supp info). Only eight plant genes were differentially expressed between green and red galls and all eight genes were downregulated in green galls (FIGS. 3C-3D). That is, high levels of dgc are associated with downregulation of eight plant genes in galls.

The seven most strongly downregulated plant genes all encode enzymes of the anthocyanin biosynthetic pathway (FIG. 3E). One gene encodes an enzyme (ACCA) that irreversibly converts acetyl-CoA to malonyl CoA, a biosynthetic precursor of multiple anthocyanins. Two genes encode anthocyanidin 3-O-glucosyltransferases (UFGT and UGT75C1), which glycosylate unstable anthocyanidins to allow their accumulation (Springob et al., 2003). Two genes encode flavonoid 3′-5′ methyltransferases (FAOMT-1, FAOMT-2), which methylate anthocyanin derivatives (Hugueney et al., 2009). Finally, two genes encode phi class glutathione S-transferase (GSTF11, GSTF12), which conjugate glutathione to anthocyanins, facilitating anthocyanin transport and stable accumulation in vacuoles (Marrs et al., 1995).

Six of these enzymes are required for final steps of anthocyanin production and deposition (FIG. 3C) and their upregulation in red galls may account for the accumulation of pigments in red galls. To test this idea, pigments from galls (FIG. 3E) were extracted and analyzed, and high levels of two pigments were identified only in red galls (FIG. 3G), the anthocyanins malvidin-3,5-diglucoside and peonidin-3,5-diglucoside (FIGS. 3G-H). Thus, the pigments present in red galls are products of enzymes in the anthocyanin biosynthetic pathway, such as those that are upregulated in red galls. The two abundant anthocyanins are produced from distinct intermediate precursor molecules (FIG. 3E), three of which were also detected in red galls (FIG. 3F, S8), and synthesis of these two anthocyanins likely requires activity of different methyltransferases and glucosyltransferases. The three pairs of glucosyltransferases, methyltransferases, and glutathione transferases upregulated in red galls may provide the specific activities required for production of these two anthocyanins.

Taken together, these observations suggest that dgc represses transcription of seven anthocyanin biosynthetic enzymes. It is not clear how dgc induces specific transcriptional changes in seven plant genes and it may act by altering activity of an upstream regulator of these plant genes.

Aphids Induce Widespread Transcriptomic Changes in Galls

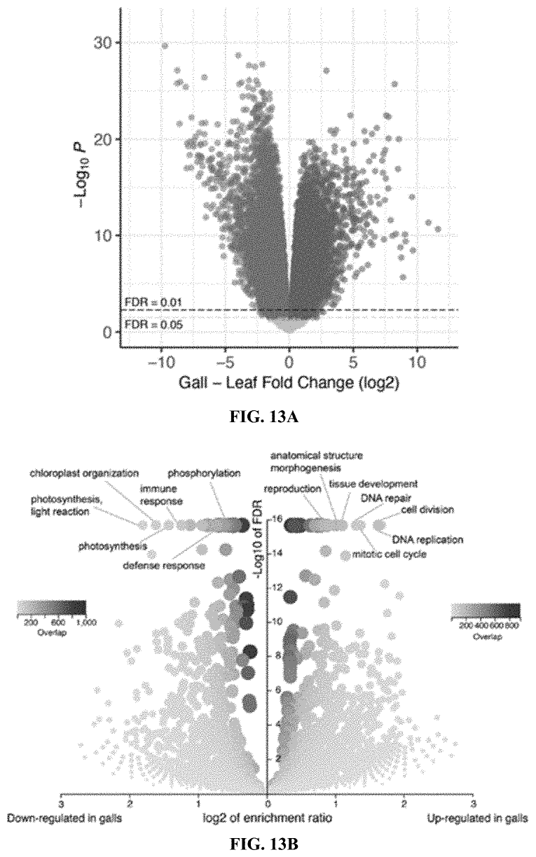

The gall color polymorphism reflects only a small part of the gall phenotype. To estimate how many plant genes are differentially expressed during gall development, differential expression analysis of plant genes in galls versus the surrounding leaf tissue was performed. Approximately 31% of plant genes were upregulated and 34% were downregulated at FDR=0.05 in galls versus leaf tissue (FIG. 13A; 27% up and 29% down in gall at FDR=0.01). Results of gene ontology analysis of up and down-regulated genes is consistent with the extensive growth of gall tissue and down regulation of chloroplasts seen in galls (FIG. 13B).

Thus, approximately 15,000 plant genes are differentially expressed in galls, representing a dramatic re-programming of plant cell biology. If other aphid effector molecules act in ways similar to dgc, which is associated with differential expression of only eight plant genes, then gall development may injection of hundreds or thousands of effector molecules.

dgc is the Founding Member of a Large Class of Novel Bicycle Genes Expressed Specifically in the Salivary Glands of Gall-Inducing Aphids

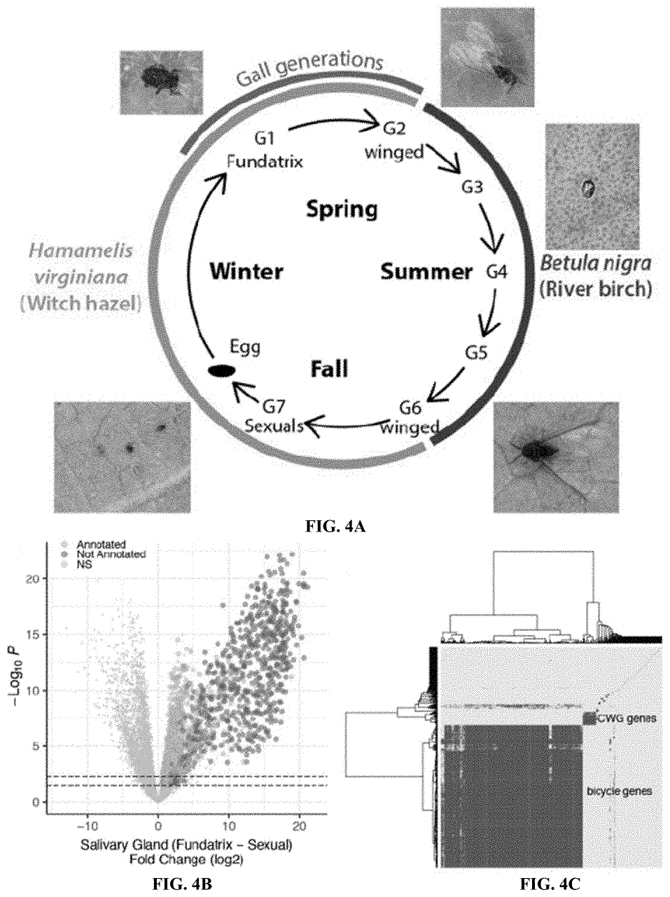

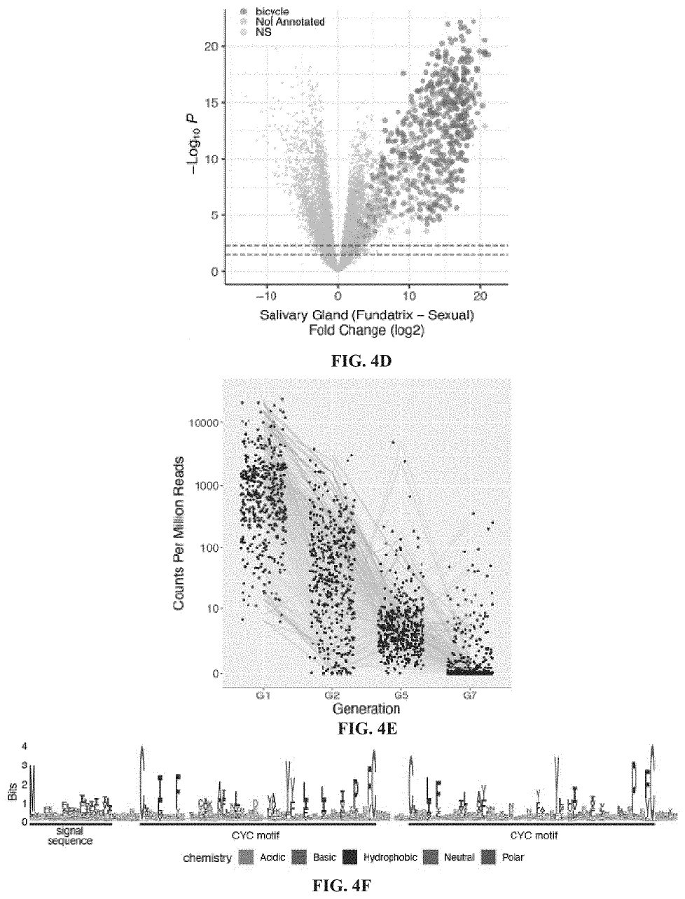

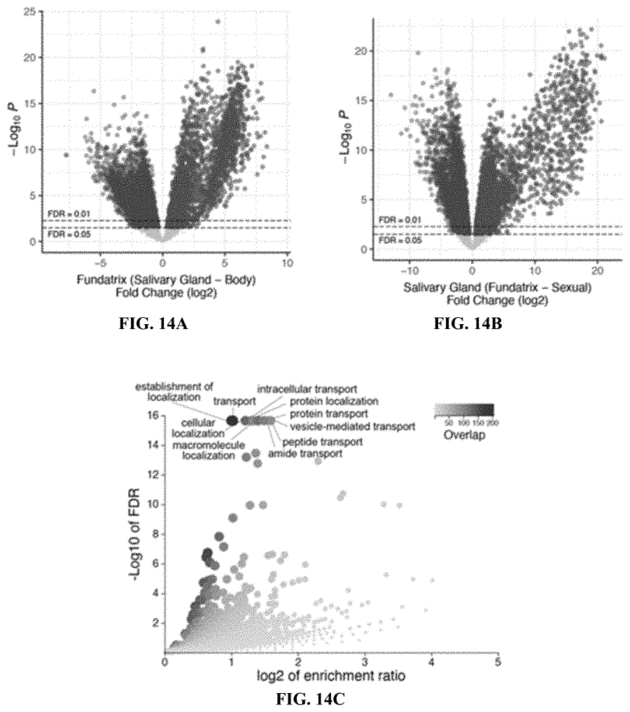

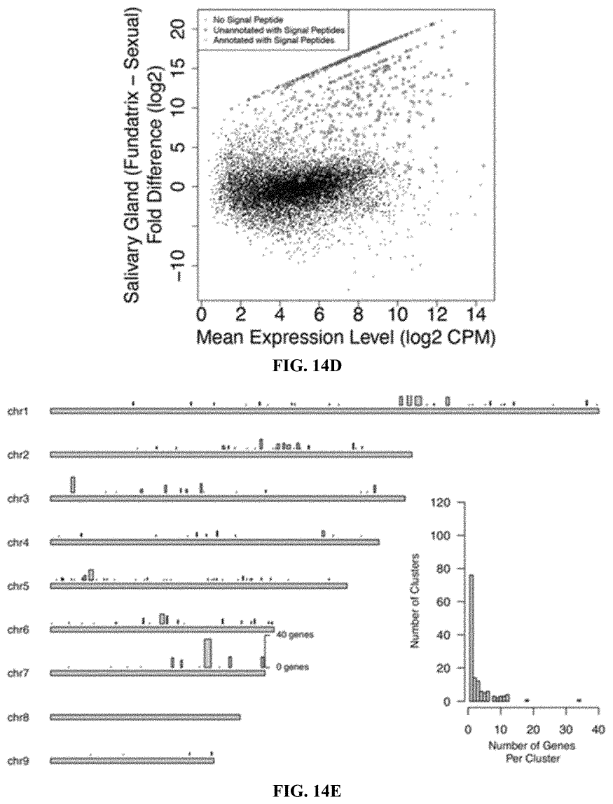

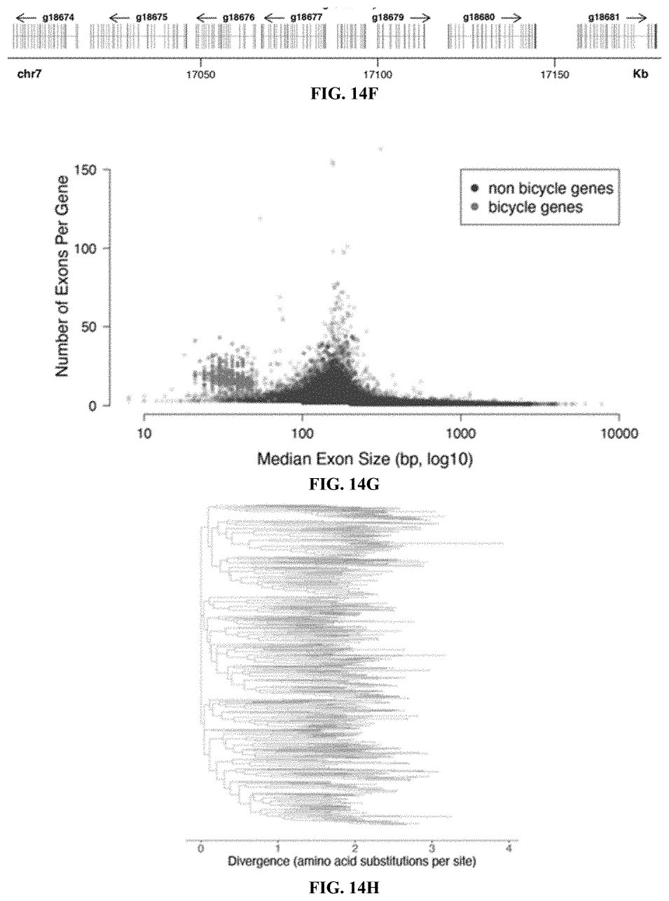

To identify additional proteins that aphids may inject into plants to contribute to gall development, the fact that only some individuals in the complex life cycle of H. cornu induce galls was exploited (FIG. 4A). It was contemplated that expression of genes required for gall formation should be enriched specifically in the salivary glands of the generations that induce galls: the fundatrix and possibly also her offspring. 3,048 genes were identified that were upregulated in fundatrix salivary glands versus the fundatrix body (FIG. 14A) and 3,427 genes upregulated in salivary glands of fundatrices, which induce galls, versus sexuals, which do not induce galls although they feed on the same host plant (FIG. 14B). Intersection of these gene sets identified 1,489 genes specifically enriched in the salivary glands of fundatrices (FIG. 4B).

Half of these genes (744) were homologous to previously identified genes, many of which had functional annotations. Gene Ontology analysis of the “annotated” genes suggests that they contribute mostly to the demands for high levels of protein secretion in fundatrix salivary glands (FIG. 14C). Most do not encode proteins with secretion signals (671; FIG. 14D) and are thus unlikely to be injected into plants. A search was conducted for homologs of genes that have been proposed as candidate gall-effector genes in other insects but no evidence was found to suggest that these classes of genes contribute to aphid gall development. Therefore, the remaining 738 unannotated genes were focused upon, which included 459 genes encoding proteins with predicted secretion signals (FIG. 14D).

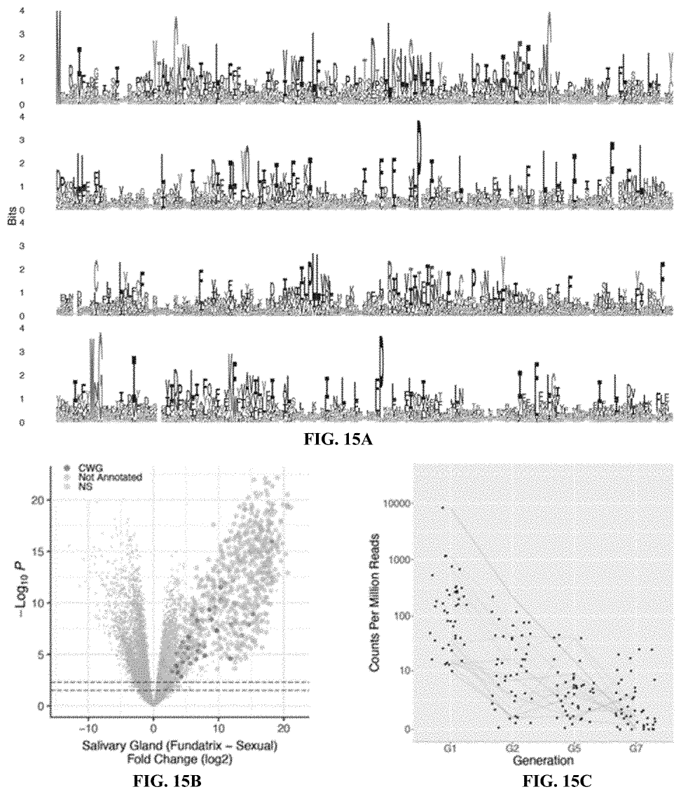

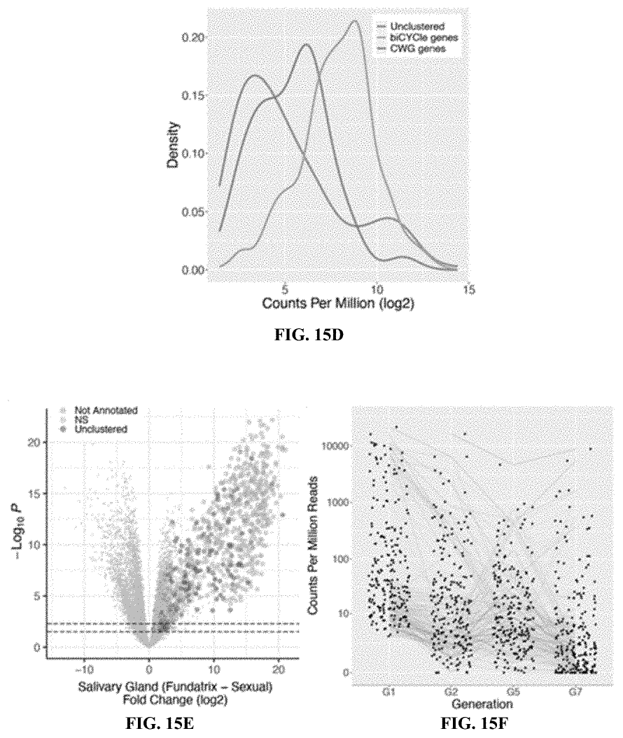

Hierarchical clustering of the unannotated genes by sequence similarity identified one large (476 genes) and one small (43 genes) cluster of related genes, and 222 genes sharing few or no homologs (FIG. 4C). Multiple sequence alignment of the protein sequences of the large and small cluster of genes revealed that both clusters encode proteins with N-terminal secretion signals, as expected for effector proteins that might be injected into plants. The small cluster encodes a divergent set of proteins containing several conserved cysteines (C) and a well conserved tryptophan (W) and glycine (G) (FIG. 15A).

Proteins encoded by the large cluster display conservation mainly of a pair of widely spaced cysteine-tyrosine-cysteine (CYC) motifs (FIG. 4F). This pair of CYC motifs led us to name these bicycle (bi-CYC-like) genes. The bicycle genes were the most strongly upregulated genes in fundatrix salivary glands (FIG. 4D, FIGS. 15B-15F) and were expressed specifically in the salivary glands of the two generations associated with galls (G1 and G2) (FIG. 4E). Many bicycle genes are found in paralog clusters throughout the H. cornu genome (FIGS. 14E and 14F) and exhibit an excess of microexons (FIGS. 14F and 14G), as was observed for dgc. In fact, dgc exhibits a pair of CYC motifs (FIG. 8) and is related to other bicycle genes.

Thus, dgc, which is associated with gall color, is the founding member of a diverse family of genes encoding secreted proteins expressed specifically in the salivary glands of gall forming generations. Bicycle genes are therefore good candidates to encode many of the molecules required to generate the extensive transcriptional changes observed in galls.

### DISCUSSION

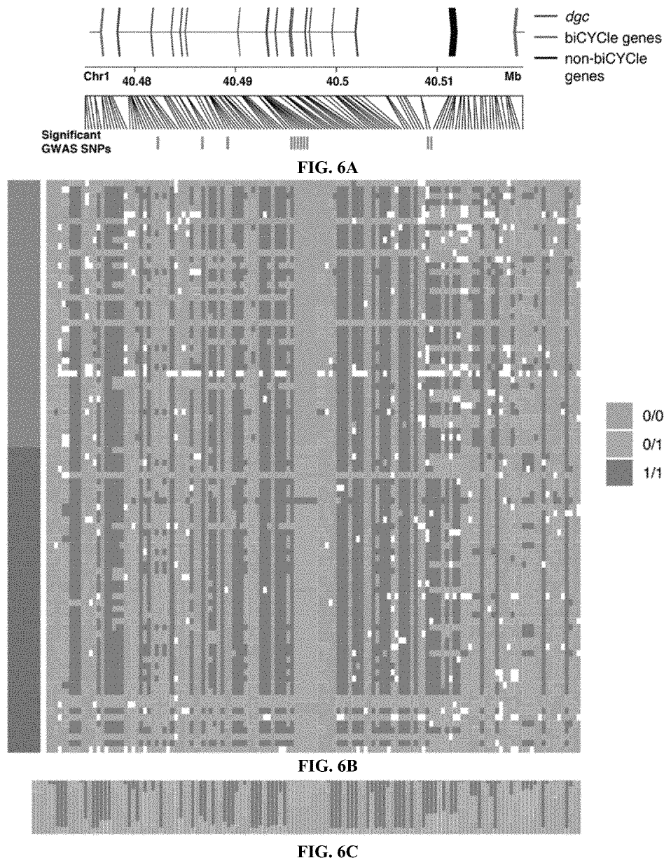

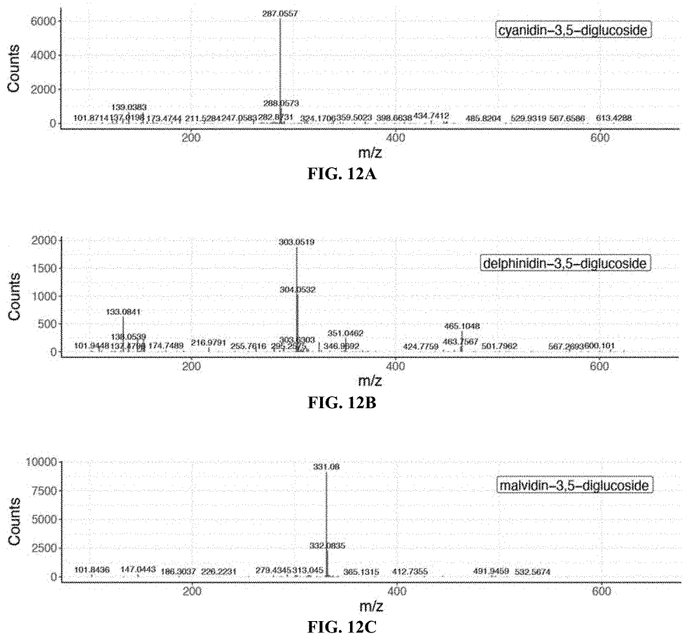

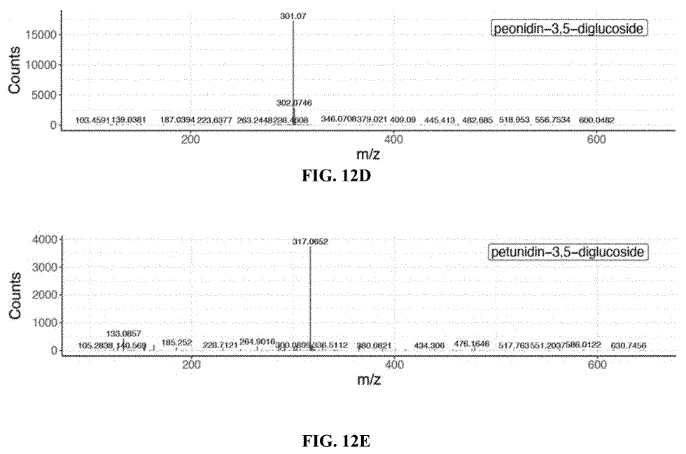

It was found that eleven derived regulatory polymorphisms at dgc in the gall forming aphid H. cornu are associated with almost complete silencing of dgc in aphid salivary glands (FIGS. 2 and 4). Low levels of dgc expression are associated specifically with high levels of seven anthocyanin biosynthetic genes and upregulation of two red-purple anthocyanins in red galls (FIGS. 5A-F, 11). Since gall color represents a small fraction of the changes involved in gall development (FIGS. 5D-5F), additional aphid genes were sought, which might encode gall effector proteins. It was found that dgc is a member of the single largest family of fundatrix salivary-gland specific secreted proteins (FIGS. 6 and 12). The most parsimonious explanation for the current observations is that bicycle gene products provide instructive molecules required for gall development. Each BICYCLE protein may regulate a unique set of specific targets in the plant.

BICYCLE Protein Functions

dgc likely encodes a protein that is deposited by aphids into gall tissue, but the mechanism by which this novel aphid protein specifically and dramatically downregulates seven anthocyanin biosynthetic genes remains to be determined. The primary sequences of DGC and other BICYCLE proteins provide few clues to their molecular mode of action. Outside of the N-terminal secretion signal, BICYCLE proteins possess no similarity with previously reported proteins and display no conserved domains that might guide functional studies. The relatively well-conserved C-Y-C motif appears to define a pair of ˜50-80 aa domains in each protein and the paired cysteines may form disulfide bonds, which is commonly observed for secreted proteins.

Methods

Imaging of Leaves and Fundatrices Inside Developing Galls

Young Hamamelis virginiana (witch hazel) leaves or leaves with early stage galls of Hormaphis cornu were fixed in Phosphate Buffered Saline (PBS: 137 mM NaCl, 2.7 mM KCl, 10 mM Na₂HPO₄, 1.8 mM KH₂PO₄, pH 7.4) containing 0.1% Triton X-100, 2% paraformaldehyde and 0.5% glutaraldehyde (paraformaldehyde and glutaraldehyde were EM grade from Electron Microscopy Services) at room temperature for two hours without agitation to prevent the disruption of the aphid stylet inserted into leaf tissue. Fixed leaves or galls were washed in PBS containing 0.1% Triton X-100, hand cut into small sections (˜10 mm2), and embedded in 7% agarose for subsequent sectioning into 0.3 mm thick sections using a Leica Vibratome (VT1000s). Sectioned plant tissue was stained with Calcofluor White (0.1 mg/mL, Sigma-Aldrich, F3543) and Congo Red (0.25 mg/mL, Sigma-Aldrich, C6767) in PBS containing 0.1% Triton X-100 with 0.5% DMSO and Escin (0.05 mg/mL, Sigma-Aldrich, E1378) at room temperature with gentle agitation for 2 days. Stained sections were washed with PBS containing 0.1% Triton X-100. Soft tissues were digested and cleared to reduce light scattering during subsequent imaging using a mixture of 0.25 mg/mL collagenase/dispase (Roche #10269638001) and 0.25 mg/mL hyaluronidase (Sigma Aldrich #H3884) in PBS containing 0.1% Triton X-100 for 5 hours at 37° C. To avoid artifacts and warping caused by osmotic shrinkage of soft tissue and agarose, samples were gradually dehydrated in glycerol (2% to 80%) and then ethanol (20% to 100%) (Ott 2008) and mounted in methyl salicylate (Sigma-Aldrich, M6752) for imaging. Serial optical sections were obtained at 2 μm intervals on a Zeiss 880 confocal microscope with a Plan-Apochromat 10×/0.45 NA objective, at 1 μm with a LD-LCI 25×/0.8 NA objective or at 0.5 μm with a Plan-Apochromat 40×/0.8 NA objective. Maximum projections of confocal stacks or rotation of images were carried out using FIJI (Schindelin et al. 2012).

Hormaphis cornu Genome Sequencing, Assembly, and Annotation

H. cornu aphids were collected from a single gall for genome sequencing. All aphids within the gall were presumed to be clonal offspring of a single fundatrix, because all H. cornu galls that were examined contain only a single fundatrix and the ostiole of the galls was closed at the time this gall was collected, so there is little chance of inter-gall migration. High molecular weight (BMW) DNA was prepared by gently grinding aphids with a plastic pestle against the inside wall of a 2 mL Eppendorf tube in 1 mL of 0.5% SDS, 200 mM Tris-HCl pH 8, 25 mM EDTA, 250 mM NaCl with 10 uL of 1 mg/mL RNAse A. Sample was incubated at 37° C. for 1 hour and then 30 uL of 10 mg/mL ProteinaseK was added and the sample was incubated for an additional 1 hr at 50° C. with gentle agitation at 300 RPM. 1 mL of Phenol:Chloroform:Isoamyl alcohol (25:24:1) was added and the sample was centrifuged at 16,000 RCF for 10 min. The supernatant was removed to a new 2 mL Eppendorf tube and the Phenol:Chloroform:Isoamyl alcohol extraction was repeated. The supernatant was removed to a new 2 mL tube and 2.5× volumes of absolute ethanol were added. The sample was centrifuged at 16,000 RCF for 15 min and then washed with fresh 70% ethanol. All ethanol was removed with a pipette and the sample was air dried for approximately 15 minutes and DNA was resuspended in 50 uL TE. This sample was sent to HudsonAlpha Institute for Biotechnology for genome sequencing.

DNA quality control, library preparation, and Chromium 10× linked read sequencing were performed by HudsonAlpha Institute for Biotechnology. Most of the mass of the BMW DNA appeared greater than approximately 50 kb on a pulsed field gel and paired end sequencing on an Illumina HiSeq×10 yielded 816 M reads. The genome was assembled using Supernova (Weisenfeld et al. 2017) using 175 M reads, which generated the best genome N50 of a range of values tested. This 10× genome consisted of 21,072 scaffolds of total length 319.352 MB. The genome scaffold N50 was 839.101 KB and the maximum scaffold length was 3.495 MB.

Dovetail Genomics was engaged to apply HiC and Chicago to generate larger scaffolds. BMW gDNA was submitted from the same sample used for 10× genome sequencing for Chicago and a separate sample of frozen aphids for HiC. The Dovetail genome consisted of 11,244 scaffolds of total length 320.34 MB with a scaffold N50 of 36.084 Mb. This genome, named hormaphis_cornu_26Sep2017_PoQx8, contains 9 main scaffolds, each longer than 17.934 Mb, which appear to represent the expected 9 chromosomes of H. cornu (Blackman and Eastop 1994). This assembly also includes the circular genome of the bacterial endosymbiont Buchnera aphidicola of 643,259 bp. BUSCO analysis (Simao et al. 2015) using the gVolante web interface (Nishimura et al., 2017) with the Arthropod gene set revealed that the genome contains 1026 of 1066 (96.25%) complete core genes and 1038 of 1066 (97.37%) partial core genes.

This genome was annotated for protein-coding genes using RNA-seq data collected from salivary glands and carcasses of many stages of the H. cornu life cycle using BRAKER (Altschul et al. 1990; Barnett et al. 2011; Camacho et al. 2009; Hoff et al. 2016, 2019; Li 2013; Lomsadze, Burns, and Borodovsky 2014; Stanke et al. 2006). To increase the efficiency of mapping RNA-seq reads for differential expression analysis, 3′ UTRs were predicted using UTRme (Radío et al. 2018). It was found that UTRme sometimes predicted UTRs within introns. Therefore, a custom R script was applied to remove such predicted UTRs. Later, after discovering the bicycle genes, all predicted bicycle genes were manually annotated, including 5′ and 3′ UTRs, in APOLLO (Lewis et al. 2002) by examining evidence from RNAseq reads aligned to the genome with the Integrative Genomics Viewer (Robinson et al. 2011; Thorvaldsdóttir, Robinson, and Mesirov 2013). It was found that the start sites of many bicycle genes were incorrectly annotated by BRAKER at a downstream methionine, inadvertently excluding predicted putative signal peptides from these genes. RNAseq evidence often supported transcription start sites that preceded an upstream methionine and these exons were corrected in APOLLO. The combined collection of 18,895 automated and 687 manually curated gene models (19,582 total) were used for all subsequent analyses of H. cornu genomic data.

Genome-Wide Association Study of Aphids Inducing Red and Green Galls

Galls produced by H. cornu were collected in the early summer (Table 1) and dissected by making a single vertical cut down the side of each gall with a razor blade to expose the aphids inside. DNA was extracted using the Zymo ZR-96 Quick gDNA kit from the foundress of each gall, because foundresses do not appear to travel between galls. Tagmentation was performed of genomic DNA derived from 47 individuals from red galls and 43 from green galls using barcoded adaptors compatible with the Illumina sequencing platform (Hennig et al. 2018). Tagmented samples were pooled without normalization, PCR amplified for 14 cycles, and sequenced on an Illumina NextSeq 500 to generated paired end 150 bp reads to an average depth of 2.9× genomic coverage. The average sequencing depth before filtering was calculated by multiplying the number of read pairs generated by SAMtools flagstat version 1.3 (Li et al. 2009) by the read length of 150 bp, then dividing by the total genome size (323,839,449 bp).

TABLE 1
Details of biological samples, sequence files, and SRA accession numbers for
genome sequencing. JRC refers to Janelia Research Campus, Ashburn VA.
These sequences can be found under the SRA BioProject PRJNA614456.

| | | | | Library | | | |
|---|---|---|---|---|---|---|---|
| | Collection | Collection | | Prep | Sequencing | # | Accession |
| Sample # | Date | Location | Species | Method | Method | Read pairs | # |
| MP2 | 15 Jun. 2016 | Morven Park, | Hormaphis | 10× | Illumina | 408,093,791 | SRR113 |
| | | Leesburg, | cornu | Chromium | PE151 | | 97593 |
| | | VA | | | | | |
| MP2 | 15 Jun. 2016 | Morven Park, | Hormaphis | Chicago | Illumina | 234,423,934 | SRR113 |
| | | Leesburg, | cornu | | PE151 | | 97592 |
| | | VA | | | | | |
| 80 | 31 May 2017 | Gap Rd, Rt. | Hormaphis | HiC | Illumina | 281,999,702 | SRR113 |
| | | 651, | cornu | | PE151 | | 97591 |
| | | Leesburg, | | | | | |
| | | VA | | | | | |
| 5552-DS- | | | Hormaphis | 10× | Illumina | 91,611,428 | |
| 0013 | | | hamamelidis | Chromiumx | PE151 | | |
| Hamamelis | Apr. 17, 2019 | JRC | Hamamelis | 10× | Illumina | 303,926,536 | SRR113 |
| virginiana | | | virginiana | Chromium | PE150 | | 97589 |
| nuclei | | | | | | | |

Principal component analysis was performed on the genome-wide polymorphism data to detect any potential population structure that might confound GWAS study. Reads were mapped using bwa mem version 0.7.17-r1188 (Li 2013) and joint genotyped using SAMtools mpileup version 1.3, with the flag -ugf followed by BCFtools call version 1.9 (Li 2011), with the flag -m. Genotype calls were then filtered for quality and missingness using BCFtools filter and view version 1.9, where only SNPs with MAF>0.05, QUAL>20, and genotyped in at least 80% of the individuals were kept. To limit the number of SNPs for computational efficiency, the SNPs were additionally thinned using VCFtools—thin version 0.1.15 to exclude any SNPs within 1000 bp of each other. PCA was performed using the snpgdsPCA function from the R package SNPRelate version 1.20.1 in R version 3.6.1 (Zheng et al. 2012).

Genome-wide association mapping was performed with these low coverage data by mapping reads with bwa mem version 0.7.17-r1188, and then calculated the likelihood of association with gall color with SAMtools mpileup version 0.1.19 and BCFtools view -vcs version 0.1.19 using BAM files as the input. Association for each SNP was measured by the likelihood-ratio test (LRT) value in the INFO field of the output VCF file, which is a one-degree association test p-value. This method calculates association likelihoods using genotype likelihoods rather than hard genotype calls, ameliorating the issue of low-confidence genotype calls resulting from low-coverage data (Li 2011). The false discovery rate was set as the Bonferroni corrected value for 0.05, which was calculated as 0.05/50,957,130 (the total number of SNPs in the genome-wide association mapping).

Enrichment and Sequencing of the Genomic Region Containing Highly Significant GWAS Hits

The low coverage GWAS identified multiple linked SNPS on chromosome 1 that were strongly associated with gall color (FIG. 2A). To identify all candidate SNPs in this genomic region and to generate higher-confidence GWAS calls, this genomic region was enriched from a library of pooled tagmented samples of fundatrix DNA from red and green galls using custom designed Arbor Bioscience MyBaits for a 800,290 bp region on chromosome 1 spanning the highest scoring GWAS SNP (40,092,625-40,892,915 bp). This enriched library was sequenced on an Illumina NextSeq 550 generating paired-end 150 bp reads and resulted in usable re-sequencing data for 48 red gall-producing individuals and 42 green gall-producing individuals, with average pre-filtered sequencing depth of 58.2×.

Reads were mapped with bwa mem version 0.7.17-r1188 and sorted bam files with SAMtools sort version 1.7, marked duplicate reads with Picard MarkDuplicates version 2.18.0 (http://broadinstitute.github.io/picard/), re-aligned indels using GATK IndelRealigner version 3.4 (McKenna et al. 2010), and called variants using SAMtools mpileup version 1.7 and BCFtools call version 1.7 (https://github.com/SAMtools/bcftools). This genotyping pipeline is available at https://github.com/YourePrettyGood/PseudoreferencePipeline (thereafter referred to as PseudoreferencePipeline). SNPs were quality filtered from the VCF file using BCFtools view version 1.7 at DP>10 and MQ>40 and merged using BCFtools merge.

For PCA analysis, the joint genotype calls were filtered for quality and missingness using BCFtools filter and view version 1.9, where only SNPs with MAF>0.05 and genotyped in at least 80% of the individuals were retained. PCA was performed using the snpgdsPCA function from the R package SNPRelate version 1.20.1 in Rstudio version 3.6.1.

Association testing was performed using PLINK version 1.90 (Purcell et al. 2007) with minor allele frequency filtered at MAF>0.2. A Hardy-Weinberg equilibrium filter was not applied because the samples were not randomly collected from nature. Red galls are rare in the population and fundatrices from red galls were oversampled to roughly match the number of fundatrices sampled from green galls. Results of the GWAS were plotted using the plotManhattan function of Sushi version 1.24.0 (Phanstiel et al. 2014).

To calculate LD for the 45 kbp region surrounding the 11 GWAS SNPs in FIG. 3B, positions chromosome 1: 40,475,000-40,520,000 were extracted from the merged VCF and retained SNPs that both exhibited MAF>0.05 and were genotyped in at least 80% of the samples using BCFtools view and filter version 1.9.

To plot LD for the entire target enrichment in FIG. 9D, the VCF were filtered for only SNPs with MAF>0.2 and genotyped in at least 80% of the samples using bcftools view and filter version 1.9, and thinned the resulting SNP set using VCFtools—thin version 0.1.15 to exclude any SNPs within 500 bp of each other. The 11 significant GWAS SNPs were further merged back using bcftools concat, since the thinning process could have removed one or more of these SNPs. SNPs were also removed in regions where the H. cornu reference genome did not align with the genome of the sister species H. hamamelidis using BEDTools intersect version 2.29.2 (Quinlan and Hall 2010).

The LD heatmaps were generated using the R packages vcfR version 1.10.0 (Knaus and Grünwald 2017), snpStats version 1.36.0 (Clayton 2019) and LDheatmap version 0.99.8 (Shin et al. 2006) in Rstudio version 3.6.1. The R code used to generate the LD heatmap figure was adapted from code provided at sfustatgen.github.io/LDheatmap/articles/vcfOnLDheatmap.html. The gene models were plotted using the plotGenes function from the R package Sushi version 1.24.0.

Lack of Evidence for Chromosomal Aberrations

To identify possible chromosomal rearrangements or transposable elements that might be linked to the GWAS SNPs, adapters were first trimmed from the H. cornu target enrichment data using Trim Galore! version 0.6.5 and cutadapt version 2.7 (Martin 2011). The trimming pipeline is available at github.com/YourePrettyGood/ParsingPipeline. Reads were then mapped the to the H. cornu reference genome with bwa mem version 0.7.17, sorted BAM files with SAMtools sort version 1.9, and marked duplicate reads with Picard MarkDuplicates version 2.22.7 (http://broadinstitute.github.io/picard/), all done with the MAP function of the PseudoreferencePipeline. The analysis includes 43 high coverage red individuals and 42 high coverage green individuals. The five individuals isolated from red galls that did not carry the associated GWAS SNPs in dgc were excluded since the genetic basis for their gall coloration is unknown.

The BAM file for each individual was subset to contain only the target enrichment region on chromosome 1 from 40,092,625 to 40,892,915 bp using SAMtools view version 1.9 and generated merged BAM file for each color using SAMtools merge. The discordant reads were then extracted from each BAM file using SAMtools view with flag -F 1286 and the percentage of discordant reads was calculated as the ratio of the number of discordant reads over the total number of mapped reads for each 5000 bp window.

To further explore the possibility that chromosomal aberrations near the GWAS signal might differ between red- and green-gall producing individuals, the mapping locations of discordant reads in the 100 kbp region near the 11 GWAS hits (40,450,000-40,550,000 bp) were plotted for red individuals, since the H. cornu reference was made from a green individual. The read ID was obtained for all the discordant reads within the 100 kbp region and extracted all occurrences of these reads from the whole genome BAM file, regardless of their mapping location. The paired-end reads were then extracted from the discordant reads BAM file using bedtools bamtofastq version 2.29.2 and used bwa mem to map these reads as single-end reads for read 1 and read 2 separately to a merged reference containing the H. cornu genome and the 343 Acyrthosiphon pisum transposable elements annotated in RepBase (Jurka 1998). Duplicates were removed and the BAM file was sorted using SAMtools rmdup and sort and determined the mapping location of all discordant reads using SAMtools view. The windows were masked from chromosome 1: 40,400,000-40,499,999 bp and 40,500,000-40,599,999 bp in the genome-wide scatter plot of discordant reads mapping because the majority of the discordant reads are expected to map to these regions and displaying their counts would obscure potential signals in the rest of the genome.

Large Scale Survey of 11 dgc SNPs Associated with Gall Color

Aphids were collected from red and green galls as described above for the GWAS study directly into Zymo DNA extraction buffer and ground with a plastic pestle. DNA was prepared using the ZR-96 Quick gDNA kit. qPCR assays and amplicon-seq assays were developed to genotype all individuals at all 11 SNPs (primers, etc below). PCR amplicon products were barcoded and samples were pooled for sequencing on an Illumina platform.

Adaptors were trimmed from amplicon reads using Trim Galore! version 0.6.5 and cutadapt version 2.7. The wrapper pipeline is available at github.com/YourePrettyGood/ParsingPipeline. Reads were mapped to a 34 kbp region of chromosome 1 of the H. cornu genome that includes the amplicon SNPs (40,477,000-40,511,000 bp) with bwa mem version 0.7.17, sorted BAM files with SAMtools sort version 1.9, and re-aligned indels using GATK IndelRealigner version 3.4. No marking of duplicates was done given the nature of amplicon sequencing data. To maximize genotyping efficiency and improve accuracy, variant calling was performed with two distinct pipelines: SAMtools mpileup version 1.7 (Li et al., 2009) plus BCFtools call version 1.7, and GATK HaplotypeCaller version 3.4. The mapping and indel re-alignment pipelines are available as part of the MAP (with flag only_bwa) and IR functions of the PseudoreferencePipeline. Using the same PseudoreferencePipeline, variant calling was performed using the MPILEUP function of BCFtools and HC function of GATK. FASTA sequences for each individual, where the genotyped SNPs were updated in the reference space, were then generated for both BCFtools and GATK variant calls using the above PseudoreferencePipeline's PSEUDOFASTA function, with flags “MPILEUP, no_markdup” and “HC, no_markdup” respectively. The BCFtools SNP updating pipeline used bcftools filter, query, and consensus version 1.9, and all sites were masked where MQ<=20 or QUAL<=26 or DP<=5. The HC SNP updating pipeline used GATK SelectVariants and FastaAlternateReferenceMaker version 3.4, and all sites where MQ<50, DP<=5, GQ<90 or RGQ<90 were masked.

The variant calls were then merged from BCFtools and GATK, as well as the qPCR genotyping results, and manually identified all missing or discrepant genotypes. These missing or discrepant genotype calls were manually curated from the indel realigned BAM files using the following criteria: for heterozygous calls, the site has to have at least two reads supporting each allele, and for homozygous calls, the site has to have at least ten reads supporting the allele and no reads supporting alternative alleles.

RNA-Seq of Salivary Glands from Aphids Inducing Red and Green Galls

Salivary glands were dissected from fundatrices isolated from green and red galls in PBS, gently pipetted the salivary glands from the dissection tray in <0.5 uL volume of PBS, and deposited glands into 3 uL of Smart-seq2 lysis buffer (0.2% Triton-X 100, 0.1 U/uL RNasin® Ribonuclease Inhibitor). RNA-seq libraries were prepared with a single-cell RNA-seq method developed by the Janelia Quantitative Genomics core facility and described previously (Cembrowski et al. 2018).

RNAseq libraries were prepared as described above for red and green gall samples except that the entire 3 uL sample of salivary glands in lysis buffer was provided as input. Barcoded samples were pooled and sequenced on an Illumina NextSeq 550. 9.0 million reads per sample were detected on average. The original oligonucleotides were replaced with the following oligonucleotides to generate unstranded reads from the entire transcript:

| | | | Purifi- |
|---|---|---|---|
| Name | Sequence | Scale | cation |
| WGB_RTr1 | TCGTCGGCAGCGTCA | 250 nm | PAGE |
| | GATGTGTATAAGAGA | | |
| | CAGTTTTTTTTTTTT | | |
| | TTTTTTTTTTTTTTT | | |
| | TTTVN | | |
| | (SEQ ID NO: 9) | | |
| WGB_PCRr1 | TCGTCGGCAGCGTCA | 250 nm | HPLC |
| | GATGTGTATAAGAGA | | |
| | CAG | | |
| | (SEQ ID NO: 10) | | |
| WGB_PCRr2 | GTCTCGTGGGCTCGG | 250 nm | HPLC |
| | AGATGTGTATAAGAG | | |
| | ACAG | | |
| | (SEQ ID NO: 11) | | |
| WGB_TS0r2 | /5Biosg/GTCTCGT | 250 nmR | RNASE |
| | GGGCTCGGAGATGTG | | |
| | TATAAGAGACArGrG | | |
| | rG | | |
| | (SEQ ID NO: 12) | | |

Samples were PCR amplified for 18 cycles and the library was prepared using ¼ of the standard Nextera XT sample size and 150 pg of cDNA.

Differential Expression Analyses of Fundatrix Salivary Glands from Red and Green Galls

All differential expression analyses for plant and aphid samples were performed in R version 3.6.1 (R Core Team 2018) and all R Notebooks are provided on Github (will be posted prior to publication). Adapters were trimmed using cutadapt version 2.7 and read counts per transcript were calculated by mapping reads to the genome with hisat2 version 2.1.0 (Kim, Langmead, and Salzberg 2015) and counting reads per gene with htseq-count version 0.12.4 (Anders, Pyl, and Huber 2015). In R, technical replicates were examined and pooled, since all replicates were very similar to each other. Exploratory data analysis was performed using interactive multidimensional scaling plots, using the command glMDSPlot from the package Glimma (Su et al. 2017), and outlier samples were excluded from subsequent analyses. Differentially expressed genes were identified using the glmQLFTest and associated functions of the package edgeR (Robinson, McCarthy, and Smyth 2009). Volcano plots were generated using the Enhanced Volcano command from the package EnhancedVolcano version 1.4.0 (Blighe, Rana, and Lewis 2020). Mean-Difference (MD) plots and multidimensional scaling plots were generated using the plotMD and plotMDS functions, respectively, from the package limma version 3.42.0 (Ritchie et al. 2015).

Hamamelis virginiana Genome Sequencing, Assembly, and Annotation

Leaves from a single tree of Hamamelis virginiana were sampled from the Janelia Research Campus forest as follows. Branches containing leaves that were less than 50% expanded were wrapped with aluminum foil and harvested after 40 hours. Leaves were cleared of obvious contamination, including aphids and other insects, and then plunged into liquid N₂. Samples were stored at −80° C. and sent to the Arizona Genomics Institute, University of Arizona on dry ice, which prepared HMW DNA from nuclei isolated from the frozen leaves. The Janelia Quantitative Genomics core facility generated a 10× chromium linked-read library from this DNA and sequenced the library on an Illumina NextSeq 550 to generate 608M linked reads.

The H. virginiana genome was assembled with the supernova commands run and mkoutput version 2.1.1, with options minsize=1000 and style=pseudohap (Weisenfeld et al. 2017). 332 M reads were used in the assembly to achieve raw coverage of 56× as recommended by the supernova instruction manual. The assembled genome was 886 Mb and the scaffold N50 was 472 Kb. BUSCO completeness analysis (Simao et al. 2015) was performed using the gVolante Web interface (Nishimura, Hara, and Kuraku 2017) using the plants database. BUSCO_v2/v3 showed 1309 of 1440 (90.9%) completely detected core genes and 1365 of 1440 (94.8%) partially detected core genes in the assembled H. virginiana reference genome.

The assembled genome reference was repeat masked with soft masking using RepeatMasker version 4.0.9 (Smit n.d.). Twenty-five RNA-seq libraries from galls and leaves were used for genome annotation. RNA-seq reads were adapter trimmed using cutadapt version 2.7 and mapped to the genome using HISAT2 version 2.1.0. Genome annotation was performed with BRAKER version 2.1.4 using the RNAseq data to provide intron hints (Altschul et al. 1990; Barnett et al. 2011; Camacho et al. 2009; Hoff et al. 2016, 2019; Li et al. 2009; Lomsadze et al. 2014; Ritchie et al. 2015; Stanke et al. 2006, 2008) and 3′ UTRs were predicted using UTRme (Radío et al. 2018).

H. virginiana RNA Extraction and RNA-Seq Library Preparation

RNA was extracted from frozen H. virginiana leaf or gall tissue as follows. Plant tissue frozen at −80° C. was placed into ZR BashingBead Lysis Tubes (pre-chilled in liquid N₂) and pulverized to a fine powder in a Talboys High Throughput homogenizer (Troemer) with minimal thawing. Powdered plant tissue was suspended in extraction buffer (100 mM Tris-HCl, pH 7.5, 25 mM EDTA, 1.5 M NaCl, 2% (w/v) Hexadecyltrimethylammonium bromide, 10% Polyvinylpyrrolidone (w/v) and 0.3% (v/v) β-mercaptoethanol) and heated to 55° C. for 8 min followed by centrifugation at 13,000×g for 5 minutes at room temperature to remove insoluble debris (Jordon-Thaden et al. 2015). Total RNA was extracted from the supernatant using the Quick-RNA Plant Miniprep Kit (Zymo Research) with the inclusion of in-column DNAse I treatment. RNA-seq libraries were prepared with the Universal Plus mRNA-Seq kit (Nugen).

Differential Expression Analysis of Red Versus Green Galls

Differential expression analysis was performed on red and green galls by collecting paired red and green gall samples from the same leaves. In total, 17 red galls and 22 green galls were collected from 17 leaves. RNA was prepared as described above for plant material and RNA-seq libraries were prepared with a single-cell RNA-seq method modified for plant material described above.

These RNAseq libraries contained on average 4.4 million mapped reads per sample. Reads were quality trimmed and mapped to the transcriptome as described above. Only genes with greater than 1 count per million in at least 15 samples were included in subsequent analyses. The expression analysis model included the effect of leaf blocking.

Gall Pigment Extraction and Analysis

Frozen gall tissue was ground to a powder under liquid nitrogen. Approximately 20 mg of ground gall tissue was suspended in 100 μL methanol (Optima grade, Fisher Scientific) and 400 μl of 5% aqueous formic acid (Optima grade, Fisher Scientific), vortexed for 30 seconds and centrifuged at 8000×g for 2 min at 10° C. The supernatant containing pigment was filtered using a 0.2 μm, 13 mm diameter PTFE syringe filter to remove debris. Colorless pellet was discarded. Authentic anthocyanin pigment standards for malvidin 3,5-diglucoside chloride (Sigma Aldrich, St. Louis, Mo., USA) and peonidin-3,5-diglucoside chloride (Carbosynth LLC, San Diego, Calif., USA) were prepared at 1 mg/mL in 5% aqueous formic acid.

Pigment separation and identification alongside standards was performed on a reverse phase C18 column (Acquity Plus BEH, 50 mm×2.1 mm, 1.7 μm particle size, Waters, Milford, Mass.) using an Agilent 1290 UHPLC coupled to an Agilent 6545 quadrupole time-of-flight mass spectrometer (Agilent Technologies, Santa Clara, Calif., USA) using an ESI probe in positive ion mode. Five μl of filtered pigment extract or a 1:100 dilution of anthocyanin standard was injected Solvent (A) consisted of 5% aqueous formic acid and (B) 1:99 water/acetonitrile acidified with 5% aqueous formic acid (v/v) (B). The gradient conditions were as follows: 1 min hold at 0% B, 4 min linear increase to 20% B, 5 min linear increase to 40% B, ramp up to 95% B in 0.1 min, hold at 95% B for 5 min, return to 0% B and hold for 0.9 min. The column flow rate was 0.3 mL/min, and the column temperature 30° C. The MS source parameters for initial anthocyanin detection were as follows: capillary=4000 V, nozzle=2000 V, gas temperature=350° C., gas flow=13 L/min, nebulizer=30 psi, sheath gas temperature=400° C., sheath gas flow=12 L/min. DAD detection at 300 nm and 520 nm, and MS scanning from 50-1700 m/z at a rate of 2 spectra per second. Iterative fragmentation, followed by targeted MS/MS experiments, were performed using a collision energy=35. Authentic standards confirmed the presence of peonidin-3,5-diglucoside and malvidin 3,5-diglucoside. The remaining anthocyanin species were identified using UV-Vis spectra, retention time relative to the other species in the sample, [M]⁺ precursor ions and aglycone fragment ions matching the respective entries in the RIKEN database (Sawada et al. 2012).

Differential Expression Analysis of Galls Versus Leaves

RNA seq was performed on 36 gall samples and 17 adjacent leaf samples. These gall samples did not overlap with the gall samples used in red versus green gall comparison described earlier. For larger galls, RNA was isolated separately from basal, medial, and apical gall regions. Libraries were sequenced on an Illumina NextSeq 550 to generate 150 bp paired-end reads with an average of 8.1 million mapped reads per sample. Only genes expressed at greater than 1 count per million in at least 18 samples were included in subsequent analysis. Only samples where there are paired gall and leaf samples from the same leaf were included and potential leaf effects were modeled in the different expression analysis.

To facilitate Gene Ontology (GO) analysis, the UniProt IDs of the differentially expressed genes were obtained by mapping the coding sequence of the H. virginana genome to the UniProt/Swiss-Prot database (Bateman 2019) using Protein-Protein BLAST 2.7.1 (Altschul et al. 1990; Camacho et al. 2009) and extracting the differentially expressed genes. The WebGestalt 2019 webtool (Liao et al. 2019) was used to perform GO analysis on the differentially expressed genes.

Differential Expression Analysis of H. cornu Organs and Life Stages

RNAseq libraries were generated for fundatrix salivary glands (N=20) and whole bodies (N=8), G2 salivary glands (N=6) and carcasses (N=3), G5 salivary glands (N=6) and carcasses (N=2), and G7 salivary glands (N=5). Libraries were generated as described above for salivary glands except that RNA samples of carcasses and whole bodies were prepared using the Arcturus PicoPure RNA Isolation Kit (Applied Biosystems). Only genes expressed with at least 1 count per million in at least 29 samples were included in subsequent analyses.

Bioinformatic Identification of Bicycle Genes in H. cornu

Genes that were upregulated specifically in the salivary glands of the fundatrix generation were prime candidates for inducing galls. Genes that were upregulated both in salivary glands of fundatrices versus sexuals (G7) and in fundatrix salivary glands were therefore identified. These differentially expressed genes were then separated into genes with and without homologs containing some functional annotation. Homologs with previous functional annotations were identified using three methods: the following were performed (1) translated query-protein (blastx) and (2) protein-protein (blastp) based homology searches using BLAST 2.7.1 (Altschul et al. 1990; Camacho et al. 2009) against the UniProt/Swiss-Prot database (Bateman 2019) (Bateman 2019), and (3) Hidden-Markov based searches with the predicted proteins using hmmscan in HMMER version 3.1b2 (Eddy 2011) against the pfam database (Finn et al. 2014). For all predicted proteins, SignalP-5.0 (Almagro Armenteros et al. 2019) was used to search for secretion signal peptides and tmhmm version 2.0 was used to search for transmembrane domains (Krogh et al. 2001). Gene Ontology analysis of genes with annotations that were enriched in fundatrix salivary glands was performed by searching for Drosophila melanogaster homologs of differentially expressed genes and using these D. melanogaster homologs as input into gene ontology analysis.

To determine whether any of these genes without detectable homologs in existing protein databases were homologous to each another, sensitive homology searches of all-against-all of these genes were performed using jackhmmer in HMMER version 3.1b2. Hierarchical clustering was performed on the quantitative results of the jackhmmer analysis by first calculating distances amongst genes with the dist function using method canberra and clustering using the hclust function with method ward.D2, both from the library stats in R (R Core Team 2018). Sequences of the clustered homologs were aligned using MAFFT version 7.419 with default parameters (Katoh 2002; Katoh and Standley 2013), trimmed aligned sequences using trimAI (Capella-Gutiérrez, Silla-Martínez, and Gabaldón 2009) with parameters -gt 0.50, and generated sequence logos by importing alignments using the functions read.alignment and ggseqlogo in the R packages seqinr (Charif and Lobry 2007) and ggseqlogo (Wagih 2017). After identification of the bicycle genes, additional bicycle genes were searched for in the entire H. cornu genome, which might not have been enriched in fundatrix salivary glands, using jackhmmer followed by hierarchical clustering to identify additional putative homologs. As described above, all these putative bicycle genes were manually annotated.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Almagro Armenteros, José Juan, Konstantinos D. Tsirigos, Casper Kaae Sønderby, Thomas Nordahl Petersen, Ole Winther, Søren Brunak, Gunnar von Heijne, and Henrik Nielsen. 2019. “SignalP 5.0 Improves Signal Peptide Predictions Using Deep Neural Networks.” Nature Biotechnology 37(4):420-23. doi: 10.1038/s41587-019-0036-z.
2. Altschul, Stephen F., Warren Gish, Webb Miller, Eugene W. Myers, and David J. Lipman. 1990. “Basic Local Alignment Search Tool.” Journal of Molecular Biology 215(3):403-10. doi: 10.1016/S0022-2836(05)80360-2.
3. Anders, S., P. T. Pyl, and W. Huber. 2015. “HTSeq—a Python Framework to Work with High-Throughput Sequencing Data.” Bioinformatics 31(2):166-69. doi: 10.1093/bioinformatics/btu638.
4. Bailey, S., Percy, D. M., Hefer, C. A., and Cronk, Q. C. B. (2015). The transcriptional landscape of insect galls: psyllid (Hemiptera) gall formation in Hawaiian Metrosideros polymorpha (Myrtaceae). BMC Genomics 16, 943-943.
5. Barnett, Derek W., Erik K. Garrison, Aaron R. Quinlan, Michael P. St\ddot{r}mberg, and Gabor T. Marth. 2011. “Bamtools: A C++ API and Toolkit for Analyzing and Managing BAM Files.” Bioinformatics 27(12):1691-92. doi: 10.1093/bioinformatics/btr174.
6. Bateman, Alex. 2019. “UniProt: A Worldwide Hub of Protein Knowledge.” Nucleic Acids Research 47(D1):D506-15. doi: 10.1093/nar/gkyl049.
7. Betancourt, A. J., and Presgraves, D. C. (2002). Linkage limits the power of natural selection in Drosophila. Proceedings of the National Academy of Sciences of the United States of America 99, 13616-13620.
8. Bilder, D., and Irvine, K. D. (2017). Taking Stock of the Drosophila Research Ecosystem. 206, 1227-1236.
9. Birky, C. W., and Walsh, J. B. (1988). Effects of linkage on rates of molecular evolution. Proceedings of the National Academy of Sciences of the United States of America 85, 6414-6418.
10. Blackman, R. L., and V. F. Eastop. 1994. Aphids on the World's Trees: An Identification and Information Guide. Wallingford: CAB International.
11. Blighe, Kevin, Sharmila Rana, and Myles Lewis. 2020. “EnhancedVolcano: Publication-Ready Volcano Plots with Enhanced Colouring and Labeling.”
12. Camacho, Christiam, George Coulouris, Vahram Avagyan, Ning Ma, Jason Papadopoulos, Kevin Bealer, and Thomas L. Madden. 2009. “BLAST+: Architecture and Applications.” BMC Bioinformatics 10:1-9. doi: 10.1186/1471-2105-10-421.
13. Capella-Gutiérrez, Salvador, José M. Silla-Martinez, and Toni Gabaldón. 2009. “TrimAl: A Tool for Automated Alignment Trimming in Large-Scale Phylogenetic Analyses.” Bioinformatics 25(15):1972-73. doi: 10.1093/bioinformatics/btp348.
14. Cembrowski, Mark S., Matthew G. Phillips, Salvatore F. DiLisio, Brenda C. Shields, Johan Winnubst, Jayaram Chandrashekar, Erhan Bas, and Nelson Spruston. 2018. “Dissociable Structural and Functional Hippocampal Outputs via Distinct Subiculum Cell Classes.” Cell 173(5):1280-1292.e18. doi: 10.1016/j.cell.2018.03.031.
15. Charif, D., and J. R. Lobry. 2007. “Seqin{R} 1.0-2: A Contributed Package to the {R} Project for Statistical Computing Devoted to Biological Sequences Retrieval and Analysis.” Pp. 207-32 in Structural approaches to sequence evolution: Molecules, networks, populations, Biological and Medical Physics, Biomedical Engineering, edited by U. Bastolla, M. Porto, H. E. Roman, and M. Vendruscolo. New York: Springer Verlag.
16. Chen, G. K., P. Marjoram, and J. D. Wall. 2008. “Fast and Flexible Simulation of DNA Sequence Data.” Genome Research 19(1):136-42. doi: 10.1101/gr.083634.108.
17. Clayton, David. 2019. “SnpStats: SnpMatrix and XSnpMatrix Classes and Methods.”
18. Cook, L. G., and Gullan, P. J. (2008). Insect, not plant, determines gall morphology in the Apiomorpha pharetrata species-group (Hemiptera: Coccoidea). Australian Journal of Entomology 47, 51-57.
19. Crespi, B., and Worobey, M. (1998). Comparative analysis of gall morphology in Australian gall thrips: The evolution of extended phenotypes. Evolution 52, 1686-1696.
20. von Dohlen, C. D., and M. B. Stoetzel. 1991. “Separation and Redescription of Hormaphis hamamelidis (Fitch 1851) and Hormaphis cornu (Shimer 1867) (Homoptera: Aphididae) on Witch-Hazel in the Eastern United States.” Proc. Entomol. Soc. Wash. 93:533-48.
21. Dodson, G. N. (1991). Control of gall morphology: tephritid gallformers (Aciurina spp.) on rabbitbrush (Chrysothamnus). Ecol. Entomol 16, 177-181.
22. von Dohlen, Carol D., Utako Kurosu, and Shigeyuki Aoki. 2002. “Phylogenetics and Evolution of the Eastern Asian-Eastern North American Disjunct Aphid Tribe, Hormaphidini (Hemiptera: Aphididae).” Molecular Phylogenetics and Evolution 23(2):257-67. doi: 10.1016/S1055-7903(02)00025-8.
23. Drozdetskiy, Alexey, Christian Cole, James Procter, and Geoffrey J. Barton. 2015. “JPred4: A Protein Secondary Structure Prediction Server.” Nucleic Acids Research 43(W1):W389-94. doi: 10.1093/nar/gkv332.
24. Dorchin, N., Hoffmann, J. H., Stirk, W. A., NovÁk, O., Strnad, M., and Van Staden, J. (2009). Sexually dimorphic gall structures correspond to differential phytohormone contents in male and female wasp larvae. Physiological Entomology 34, 359-369.
25. Eddy, Sean R. 2011. “Accelerated Profile HMM Searches.” PLoS Computational Biology 7(10). doi: 10.1371/journal.pcbi.1002195.
26. Elzinga, D. A., and Jander, G. (2013). The role of protein effectors in plant-aphid interactions. Current Opinion in Plant Biology 16, 451-456.
27. Finn, Robert D., Alex Bateman, Jody Clements, Penelope Coggill, Ruth Y. Eberhardt, Sean R. Eddy, Andreas Heger, Kirstie Hetherington, Liisa Holm, Jaina Mistry, Erik L. L. Sonnhammer, John Tate, and Marco Punta. 2014. “Pfam: The Protein Families Database.” Nucleic Acids Research 42(D1):222-30. doi: 10.1093/nar/gkt1223.
28. Hennig, Bianca P., Lars Velten, Ines Racke, Chelsea Szu Tu, Matthias Thoms, Vladimir Rybin, Hüseyin Besir, Kim Remans, and Lars M. Steinmetz. 2018. “Large-Scale Low-Cost NGS Library Preparation Using a Robust Tn5 Purification and Tagmentation Protocol.” G3: Genes, Genomes, Genetics 8(1):79-89. doi: 10.1534/g3.117.300257.
29. Hoff, Katharina J., Simone Lange, Alexandre Lomsadze, Mark Borodovsky, and Mario Stanke. 2016. “BRAKER1: Unsupervised RNA-Seq-Based Genome Annotation with GeneMark-ET and AUGUSTUS.” Bioinformatics 32(5):767-69. doi: 10.1093/bioinformatics/btv661.
30. Hoff, Katharina J., Alexandre Lomsadze, Mark Borodovsky, and Mario Stanke. 2019. Whole-Genome Annotation with BRAKER. Vol. 1962.
31. Hogenhout, S. A., and Bos, J. I. B. (2011). Effector proteins that modulate plant-insect interactions. Current Opinion in Plant Biology 14, 422-428.
32. Hugueney, P., Provenzano, S., Verriés, C., Ferrandino, A., Meudec, E., Batelli, G., Merdinoglu, D., Cheynier, V., Schubert, A., and Ageorges, A. (2009). A novel cation-dependent o-methyltransferase involved in anthocyanin methylation in grapevine. Plant Physiology 150, 2057-2070.
33. Inbar, M., A. Eshel, and D. Wool. 1995. “Interspecific Competition among Phloem-Feeding Insects Mediated by Induced Host-Plant Sinks.” Ecology 76(5):1506-15. doi: 10.2307/1938152.
34. Jurka, Jerzy. 1998. “Repeats in Genomic DNA: Mining and Meaning.” Current Opinion in Structural Biology 8(3):333-37. doi: 10.1016/50959-440X(98)80067-5.
35. Kaloshian, I., and Walling, L. L. (2016). Hemipteran and dipteran pests: Effectors and plant host immune regulators. Journal of Integrative Plant Biology 58, 350-361.
36. Katoh, K. 2002. “MAFFT: A Novel Method for Rapid Multiple Sequence Alignment Based on Fast Fourier Transform.” Nucleic Acids Research 30(14):3059-66. doi: 10.1093/nar/gkf436.
37. Katoh, Kazutaka, and Daron M. Standley. 2013. “MAFFT Multiple Sequence Alignment Software Version 7: Improvements in Performance and Usability.” Molecular Biology and Evolution 30(4):772-80. doi: 10.1093/molbev/mst010.
38. Keightley, Peter D., Rob W. Ness, Daniel L. Halligan, and Penelope R. Haddrill. 2014. “Estimation of the Spontaneous Mutation Rate per Nucleotide Site in a Drosophila Melanogaster Full-Sib Family.” Genetics 196(1):313-20. doi: 10.1534/genetics.113.158758.
39. Kim, Daehwan, Ben Langmead, and Steven L. Salzberg. 2015. “HISAT: A Fast Spliced Aligner with Low Memory Requirements.” Nature Methods 12(4):357-60. doi: 10.1038/nmeth.3317.
40. Kimura, Motoo. 1968. “Evolutionary Rate at the Molecular Level.” Nature 217(5129):624-26. doi: 10.1038/217624a0.
41. Knaus, Brian J., and Niklaus J. Grünwald. 2017. “VCFR: A Package to Manipulate and Visualize Variant Call Format Data in R.” Molecular Ecology Resources 17(1):44-53. doi: 10.1111/1755-0998.12549.
42. Krogh, Anders, Björn Larsson, Gunnar Von Heijne, and Erik L. L. Sonnhammer. 2001. “Predicting Transmembrane Protein Topology with a Hidden Markov Model: Application to Complete Genomes.” Journal of Molecular Biology 305(3):567-80. doi: 10.1006/jmbi.2000.4315.
43. Larson, K. C., and Whitham, T. G. (1991). Manipulation of food resources by a gall-forming aphid: the physiology of sink-source interactions. Oecologia 88, 15-21.
44. Leatherdale, D. (1955). Plant hyperplasia induced with a cell-free insect extract. Nature 175, 553-554.
45. Lewis, I. F., and Walton, L. (1947). Initiation of the cone gall of witch hazel. Science 106, 419-420.
46. Lewis, I. F., and Walton, L. (1958). Gall-formation on Hamamelis virginiana resulting from material injected by the aphid Hormaphis hamamelidis. Trans. Am. Microscop. Soc. 77, 146-200.
47. Lewis, S. E., S. M. Searle, N. Harris, M. Gibson, V. Lyer, J. Richter, C. Wiel, L. Bayraktaroglir, E. Birney, M. A. Crosby, J. S. Kaminker, B. B. Matthews, S. E. Prochnik, C. D. Smithy, J. L. Tupy, G. M. Rubin, S. Misra, C. J. Mungall, and M. E. Clamp. 2002. “Apollo: A Sequence Annotation Editor.” Genome Biology 3(12):1-14. doi: 10.1186/gb-2002-3-12-research0082.
48. Li, Heng. 2011. “A Statistical Framework for SNP Calling, Mutation Discovery, Association Mapping and Population Genetical Parameter Estimation from Sequencing Data.” Bioinformatics 27(21):2987-93. doi: 10.1093/bioinformatics/btr509.
49. Li, Heng. 2013. “Aligning Sequence Reads, Clone Sequences and Assembly Contigs with BWA-MEM.” ArXiv 1303.3997.
50. Li, Heng, Bob Handsaker, Alec Wysoker, Tim Fennell, Jue Ruan, Nils Homer, Gabor Marth, Goncalo Abecasis, and Richard Durbin. 2009. “The Sequence Alignment/Map Format and SAMtools.” Bioinformatics 25(16):2078-79. doi: 10.1093/bioinformatics/btp352.
51. Liao, Yuxing, Jing Wang, Eric J. Jaehnig, Zhiao Shi, and Bing Zhang. 2019. “WebGestalt 2019: Gene Set Analysis Toolkit with Revamped UIs and APIs.” Nucleic Acids Research 47(W1):W199-205. doi: 10.1093/nar/gkz401.
52. Lomsadze, Alexandre, Paul D. Burns, and Mark Borodovsky. 2014. “Integration of Mapped RNA-Seq Reads into Automatic Training of Eukaryotic Gene Finding Algorithm.” Nucleic Acids Research 42(15):1-8. doi: 10.1093/nar/gku557.
53. Martin, Marcel. 2011. “Cutadapt Removes Adapter Sequences from High-Throughput Sequencing Reads.” EMBnet.Journal 17(1):10-10. doi: 10.14806/ej.17.1.200.
54. Mani, M. S. (1964). Ecology of Plant Galls (The Hague, The Netherlands: Springer-Science+Business Media, B.V.).
55. Marrs, K. A., Alfenito, M. R., Lloyd, A. M., and Walbot, V. (1995). A glutathione S-transferase involved in vacuolar transfer encoded by the maize gene Bronze-2. Nature 375, 397-400.
56. Martin, J. P. (1938). Stem galls of sugar cane induced with an insect extract. Hawaiian Planters' Record 42, 129-134.
57. McCalla, D. R., Genthe, M. K., and Hovanitz, W. (1962). Chemical Nature of an Insect Gall Growth-Factor. Plant Physiol 37, 98-103.
58. McDonald, J. H., and Kreitman, M. (1991). Adaptive Protein Evolution at the Adh Locus in Drosophila. Nature 351, 652-654.
59. McDonald, John H., and Martin Kreitman. 1991. “Adaptive Protein Evolution at the Adh Locus in Drosophila.” Nature 351(6328):652-54. doi: 10.1038/351652a0.
60. McKenna, Aaron, Matthew Hanna, Eric Banks, Andrey Sivachenko, Kristian Cibulskis, Andrew Kernytsky, Kiran Garimella, David Altshuler, Stacey Gabriel, Mark Daly, and Mark A. DePristo. 2010. “The Genome Analysis Toolkit: A MapReduce Framework for Analyzing next-Generation DNA Sequencing Data.” Genome Research 20(9):1297-1303. doi: 10.1101/gr.107524.110.
61. Metzger, Meredith B., Jonathan N. Pruneda, Rachel E. Klevit, and Allan M. Weissman. 2014. “RING-Type E3 Ligases: Master Manipulators of E2 Ubiquitin-Conjugating Enzymes and Ubiquitination.” Biochimica et Biophysica Acta—Molecular Cell Research 1843 (1):47-60. doi: 10.1016/j.bbamcr.2013.05.026.
62. Moran, N. A., and Whitham, T. G. (1990). Differential Colonization of Resistant and Susceptible Host Plants: Pemphigus and Populus. Ecology 71, 1059-1067.
63. Nabity, P. D., Haus, M. J., Berenbaum, M. R., and DeLucia, E. H. (2013). Leaf-galling phylloxera on grapes reprograms host metabolism and morphology. Proceedings of the National Academy of Sciences of the United States of America 110, 16663-16668.
64. Nishimura, Osamu, Yuichiro Hara, and Shigehiro Kuraku. 2017. “GVolante for Standardizing Completeness Assessment of Genome and Transcriptome Assemblies.” Bioinformatics 33(22):3635-37. doi: 10.1093/bioinformatics/btx445.
65. Obbard, D. J., Jiggins, F. M., Halligan, D. L., and Little, T. J. (2006). Natural selection drives extremely rapid evolution in antiviral RNAi genes. Current Biology 16, 580-585.
66. Ott, Swidbert R. 2008. “Confocal Microscopy in Large Insect Brains: Zinc-Formaldehyde Fixation Improves Synapsin Immunostaining and Preservation of Morphology in Whole-Mounts.” Journal of Neuroscience Methods 172(2):220-30. doi: 10.1016/j.jneumeth.2008.04.031.
67. Pavlidis, Pavlos, Daniel J̆ivković, Alexandros Stamatakis, and Nikolaos Alachiotis. 2013. “SweeD: Likelihood-Based Detection of Selective Sweeps in Thousands of Genomes.” Molecular Biology and Evolution 30(9):2224-34. doi: 10.1093/molbev/mst112.
68. Papkou, A., Guzella, T., Yang, W., Koepper, S., Pees, B., Schalkowski, R., Barg, M. C., Rosenstiel, P. C., Teotónio, H., and Schulenburg, H. (2019). The genomic basis of red queen dynamics during rapid reciprocal host-pathogen coevolution. Proceedings of the National Academy of Sciences of the United States of America 116, 923-928.
69. Parr, T. (1940). Asterolecanium variolosum Ratzeburg, a gall-forming coccid, and its effect upon the host trees. Yale University School of Forestry Bulletin 46, 1-49.
70. Paterson, S., Vogwill, T., Buckling, A., Benmayor, R., Spiers, A. J., Thomson, N. R., Quail, M., Smith, F., Walker, D., Libberton, B., et al. (2010). Antagonistic coevolution accelerates molecular evolution. Nature 464, 275-278.
71. Pavlidis, P., Z̆ivkovic, D., Stamatakis, A., and Alachiotis, N. (2013). SweeD: Likelihood-Based Detection of Selective Sweeps in Thousands of Genomes. Molecular Biology & Evolution 30, 2224-2234.
72. Plumb, G. H. (1953). The formation and development of the Norway Spruce gall caused by Adelges abietis L. Bulletin of the Connecticut Experiment Station 566, 1-77.
73. Phanstiel, Douglas H., Alan P. Boyle, Carlos L. Araya, and Michael P. Snyder. 2014. “Sushi.R: Flexible, Quantitative and Integrative Genomic Visualizations for Publication-Quality Multi-Panel Figures.” Bioinformatics 30(19):2808-10. doi: 10.1093/bioinformatics/btu379.
74. Price, Morgan N., Paramvir S. Dehal, and Adam P. Arkin. 2010. “FastTree 2—Approximately Maximum-Likelihood Trees for Large Alignments.” PLOS ONE 5(3):e9490. doi: 10.1371/journal.pone.0009490.
75. Purcell, Shaun, Benjamin Neale, Kathe Todd-Brown, Lori Thomas, Manuel A. R. Ferreira, David Bender, Julian Maller, Pamela Sklar, Paul I. W. De Bakker, Mark J. Daly, and Pak C. Sham. 2007. “PLINK: A Tool Set for Whole-Genome Association and Population-Based Linkage Analyses.” American Journal of Human Genetics 81(3):559-75. doi: 10.1086/519795.
76. Quinlan, Aaron R., and Ira M. Hall. 2010. “BEDTools: A Flexible Suite of Utilities for Comparing Genomic Features.” Bioinformatics 26(6):841-42. doi: 10.1093/bioinformatics/btq033.
77. R Core Team. 2018. “R: A Language and Environment for Statistical Computing.”
78. Radío, Santiago, Rafael Sebastián Fort, Beatriz Garat, José Sotelo-Silveira, and Pablo Smircich. 2018. “UTRme: A Scoring-Based Tool to Annotate Untranslated Regions in Trypanosomatid Genomes.” Frontiers in Genetics 9(December):671-671. doi: 10.3389/fgene.2018.00671.
79. Rahman, Atif, Ingileif Hallgrímsdóttir, Michael Eisen, and Lior Pachter. 2018. “Association Mapping from Sequencing Reads Using K-Mers.” ELife 7:e32920. doi: 10.7554/eLife.32920.
80. Rehill, B. J., and Schultz, J. C. (2001). Hormaphis hamamelidis and gall size: a test of the plant vigor hypothesis. Oikos 95, 94-104.
81. Ritchie, Matthew E., Belinda Phipson, Di Wu, Yifang Hu, Charity W. Law, Wei Shi, and Gordon K. Smyth. 2015. “Limma Powers Differential Expression Analyses for RNA-Sequencing and Microarray Studies.” Nucleic Acids Research 43(7):e47-e47. doi: 10.1093/nar/gkv007.
82. Robinson, James T., Helga Thorvaldsdóttir, Wendy Winckler, Mitchell Guttman, Eric S. Lander, Gad Getz, and Jill P. Mesirov. 2011. “Integrative Genomics Viewer.” Nature Biotechnology 29(1):24-26. doi: 10.1038/nbt0111-24.
83. Robinson, Mark D., Davis J. McCarthy, and Gordon K. Smyth. 2009. “EdgeR: A Bioconductor Package for Differential Expression Analysis of Digital Gene Expression Data.” Bioinformatics 26(1):139-40. doi: 10.1093/bioinformatics/btp616.
84. Sawada, Yuji, Ryo Nakabayashi, Yutaka Yamada, Makoto Suzuki, Muneo Sato, Akane Sakata, Kenji Akiyama, Tetsuya Sakurai, Fumio Matsuda, Toshio Aoki, Masami Yokota Hirai, and Kazuki Saito. 2012. “RIKEN Tandem Mass Spectral Database (ReSpect) for Phytochemicals: A Plant-Specific MS/MS-Based Data Resource and Database.” Phytochemistry 82:38-45. doi: 10.1016/j.phytochem.2012.07.007.
85. Schindelin, Johannes, Ignacio Arganda-Carreras, Erwin Frise, Verena Kaynig, Mark Longair, Tobias Pietzsch, Stephan Preibisch, Curtis Rueden, Stephan Saalfeld, Benjamin Schmid, Jean-Yves Yves Tinevez, Daniel James White, Volker Hartenstein, Kevin Eliceiri, Pavel Tomancak, and Albert Cardona. 2012. “Fiji: An Open-Source Platform for Biological-Image Analysis.” Nat Methods 9(7):676-82. doi: 10.1038/nmeth.2019.
86. Schaller, A. (2008). Induced plant resistance to herbivory (Springer).
87. Schultz, J. C., Edger, P. P., Body, M. J. A., and Appel, H. M. (2019). A galling insect activates plant reproductive programs during gall development. Scientific Reports 9, 1833-1833.
88. Shih, T. H., Lin, S. H., Huang, M. Y., Sun, C. W., and Yang, C. M. (2018). Transcriptome profile of cup-shaped galls in Litsea acuminata leaves. PLoS ONE 13, 1-14.
89. Shin, Ji-Hyung, Sigal Blay, Jinko Graham, and Brad McNeney. 2006. “LDheatmap: An R Function for Graphical Display of Pairwise Linkage Disequilibria Between Single Nucleotide Polymorphisms.” Journal of Statistical Software 16(Code Snippet 3). doi: 10.18637/jss.v016.c03.
90. Springob, K., Nakajima, J.-I., Yamazaki, M., and Saito, K. (2003). Recent advances in the biosynthesis and accumulation of anthocyanins.
91. Stern, D. L. (1995). Phylogenetic evidence that aphids rather than plants, determine gall morphology. Proceedings of Royal Society London B 260, 85-89.
92. Stone, G. N., and Cook, J. M. (1998). The structure of cynipid oak galls: patterns in the evolution of an extended phenotype. Proceedings of the Royal Society, London Series B 265, 979-988.
93. Stuart, J. (2015). Insect effectors and gene-for-gene interactions with host plants. Current Opinion in Insect Science 9, 56-61.
94. Suzuki, H., Yokokura, J., Ito, T., Arai, R., Yokoyama, C., Toshima, H., Nagata, S., Asami, T., and Suzuki, Y. (2014). Biosynthetic pathway of the phytohormone auxin in insects and screening of its inhibitors. Insect Biochemistry and Molecular Biology 53, 66-72
95. Simao, Felipe A., Robert M. Waterhouse, Panagiotis Ioannidis, Evgenia V. Kriventseva, and Evgeny M. Zdobnov. 2015. “BUSCO: Assessing Genome Assembly and Annotation Completeness with Single-Copy Orthologs.” Bioinformatics 31(19):3210-12. doi: 10.1093/bioinformatics/btv351.
96. Smit, A. F. A., Hubley, R., Green, P. n.d. RepeatMasker Open-4.0.
97. Stanke, Mario, Mark Diekhans, Robert Baertsch, and David Haussler. 2008. “Using Native and Syntenically Mapped CDNA Alignments to Improve de Novo Gene Finding.” Bioinformatics 24(5):637-44. doi: 10.1093/bioinformatics/btn013.
98. Stanke, Mario, Oliver Schöffmann, Burkhard Morgenstern, and Stephan Waack. 2006. “Gene Prediction in Eukaryotes with a Generalized Hidden Markov Model That Uses Hints from External Sources.” BMC Bioinformatics 7:1-11. doi: 10.1186/1471-2105-7-62.
99. Su, Shian, Charity W. Law, Casey Ah-Cann, Marie-Liesse Asselin-Labat, Marnie E. Blewitt, and Matthew E. Ritchie. 2017. “Glimma: Interactive Graphics for Gene Expression Analysis.” Bioinformatics 33(13):2050-52.
100. Takeda, S., Yoza, M., Amano, T., Ohshima, I., Hirano, T., Sato, M. H., Sakamoto, T., and Kimura, S. (2019). Comparative transcriptome analysis of galls from four different host plants suggests the molecular mechanism of gall development. PloS One 14, e0223686-e0223686.
101. Tanaka, Y., Okada, K., Asami, T., and Suzuki, Y. (2013). Phytohormones in Japanese Mugwort Gall Induction by a Gall-Inducing Gall Midge. Bioscience, Biotechnology and Biochemistry 77, 1942-1948.
102. Thorvaldsdóttir, Helga, James T. Robinson, and Jill P. Mesirov. 2013. “Integrative Genomics Viewer (IGV): High-Performance Genomics Data Visualization and Exploration.” Briefings in Bioinformatics 14(2):178-92. doi: 10.1093/bib/bbs017.
103. Tooker, J. F., and Helms, A. M. (2014). Phytohormone Dynamics Associated with Gall Insects, and their Potential Role in the Evolution of the Gall-Inducing Habit. Journal of Chemical Ecology 40, 742-753.
104. Wagih, Omar. 2017. “Ggseqlogo: A Versatile R Package for Drawing Sequence Logos.” Bioinformatics 33(22):3645-47. doi: 10.1093/bioinformatics/btx469.
105. Weisenfeld, Neil I., Vijay Kumar, Preyas Shah, Deanna M. Church, and David B. Jaffe. 2017. “Direct Determination of Diploid Genome Sequences.” Genome Research 27(5):757-67. doi: 10.1101/gr.235812.118.
106. Yamaguchi, H., Tanaka, H., Hasegawa, M., Tokuda, M., Asami, T., and Suzuki, Y. (2012). Phytohormones and willow gall induction by a gall-inducing sawfly. New Phytologist 196, 586-595.
107. Yang, Z., and Bielawski, J. P. (2000). Statistical methods for detecting molecular adaptation. Trends in Ecology & Evolution 15, 496-503.
108. Yang, Z. 2007. “PAML 4: Phylogenetic Analysis by Maximum Likelihood.” Molecular Biology and Evolution 24(8):1586-91. doi: 10.1093/molbev/msm088.
109. Yu, Guangchuang. 2020. “Using Ggtree to Visualize Data on Tree-Like Structures.” Current Protocols in Bioinformatics 69(1): e96. doi: 10.1002/cpbi 0.96.
110. Zhao, Chaoyang, Lucio Navarro Escalante, Hang Chen, Thiago R. Benatti, Jiaxin Qu, Sanjay Chellapilla, Robert M. Waterhouse, David Wheeler, Martin N. Andersson, Riyue Bao, Matthew Batterton, Susanta K. Behura, Kerstin P. Blankenburg, Doina Caragea, James C. Carolan, Marcus Coyle, Mustapha El-Bouhssini, Liezl Francisco, Markus Friedrich, Navdeep Gill, Tony Grace, Cornelis J. P. Grimmelikhuijzen, Yi Han, Frank Hauser, Nicolae Herndon, Michael Holder, Panagiotis Ioannidis, Laronda Jackson, Mehwish Javaid, Shalini N. Jhangiani, Alisha J. Johnson, Divya Kalra, Viktoriya Korchina, Christie L. Kovar, Fremiet Lara, Sandra L. Lee, Xuming Liu, Christer Löfstedt, Robert Mata, Tittu Mathew, Donna M. Muzny, Swapnil Nagar, Lynne V. Nazareth, Geoffrey Okwuonu, Fiona Ongeri, Lora Perales, Brittany F. Peterson, Ling Ling Pu, Hugh M. Robertson, Brandon J. Schemerhorn, Steven E. Scherer, Jacob T. Shreve, Denard Simmons, Subhashree Subramanyam, Rebecca L. Thornton, Kun Xue, George M. Weissenberger, Christie E. Williams, Kim C. Worley, Dianhui Zhu, Yiming Zhu, Marion O. Harris, Richard H. Shukle, John H. Werren, Evgeny M. Zdobnov, Ming Shun Chen, Susan J. Brown, Jeffery J. Stuart, and Stephen Richards. 2015. “A Massive Expansion of Effector Genes Underlies Gall-Formation in the Wheat Pest Mayetiola destructor.” Current Biology 25(5):613-20. doi: 10.1016/j.cub.2014.12.057.
111. Zhao, Chaoyang, Claude Rispe, and Paul D. Nabity. 2019. “Secretory RING Finger Proteins Function as Effectors in a Grapevine Galling Insect.” BMC Genomics 20(1):1-12. doi: 10.1186/s12864-019-6313-x.
112. Zheng, X., D. Levine, J. Shen, S. M. Gogarten, C. Laurie, and B. S. Weir. 2012. “A High-Performance Computing Toolset for Relatedness and Principal Component Analysis of SNP Data.” Bioinformatics 28(24):3326-28. doi: 10.1093/bioinformatics/bts606.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A polynucleotide molecule, comprising a nucleic acid sequence having at least about 75% identity to SEQ ID NO: 3, SEQ ID NO: 4, SEQ ID NO: 7, or SEQ ID NO: 8.

2. The polynucleotide molecule of claim 1, comprising a nucleic acid sequence having the sequence of SEQ ID NO: 3, SEQ ID NO: 4, SEQ ID NO: 7, SEQ ID NO: 8, or a functional fragment thereof.

3. The polynucleotide molecule of claim 1, optimized for expression in Arabidopsis thaliana and comprising the sequence of SEQ ID NO: 7.

4. The polynucleotide molecule of claim 1, optimized for expression in Oryza sativa and comprising the sequence of SEQ ID NO: 8.

5. A vector, comprising the polynucleotide molecule of claim 1.

6. A vector, comprising the polynucleotide molecule of claim 2.

7. A composition, comprising the vector of claim 5.

8. A composition, comprising: the vector according to claim 6.

9. A polypeptide molecule, comprising the amino acid sequence of SEQ ID NO: 5, SEQ ID NO: 6, or a functional fragment thereof.

10. A composition, comprising: the polypeptide molecule of claim 9, formulated for administration to a cell.

11. A method of modifying a cell, comprising administering to the cell a vector comprising or a polypeptide encoded by the polynucleotide molecule according to claim 1.

12. The method of claim 11, wherein the cell is selected from the group consisting of a plant cell, an animal cell, a fungal cell, and a bacterial cell.

13. A method of modulating production of anthocyanins or their precursors in a plant, comprising: administering to the plant a vector comprising or a polypeptide encoded by the polynucleotide according to claim 1.

14. The method of claim 13, wherein the production of anthocyanins or their precursors is reduced.

15. The method of claim 13, wherein the anthocyanins are selected from peonidin-3,5-diglucoside and malvidin-3,5-diglucoside.

16. The method of claim 14, wherein the precursors are elected from cynidin-3,5-diglucoside, delphinidin-3,5-diglucoside, and petunidin-3,5-diglucoside.

17. A method of modulating expression of a gene in a cell, comprising: administering to the cell a vector comprising or a polypeptide encoded by the polynucleotide molecule according claim 1, wherein the gene is selected from: FAOMT-1, FAOMT-2, GSTF11, GSTF12, UGT75C1, UFGT, ACCA, and combinations thereof.

18. The method of claim 17, wherein the cell is selected from the group consisting of a plant cell, an animal cell, a fungal cell, and a bacterial cell.

19. The method of claim 17, wherein the gene expression is downregulated.
